(19) United States
(12) Patent Application Publication
(10) Pub. No.: US 2025/0082229 A1
(43) Pub. Date: Mar. 13, 2025

(54) A Machine Learning Pipeline for Highly-Sensitive Assessment of Rotator Cuff Function

(71) Applicant: The Regents of the University of California, Oakland, CA (US)

(72) Inventors: David Darevsky, San Francisco, CA (US); Brian Feeley, San Francisco, CA (US); Xuhui Liu, Washington, DC (US); Michael Davies, San Francisco, CA (US)

(21) Appl. No.: 18/729,383

(22) PCT Filed: Jan. 25, 2023
(86) PCT No.: PCT/US2023/011543

§ 371 Date: Jul. 16, 2024

Related U.S. Application Data
(60) Provisional application No. 63/303,865, filed on Jan. 27, 2022.

(51) Int. Cl. A61B 5/11; G01S 17/08; G06V 10/62; G06V 10/82; G06V 40/16; G06V 40/20

(52) U.S. Cl. CPC A61B 5/1118; A61B 5/1128; G01S 17/08; G06V 10/62; G06V 10/82; G06V 40/171; G06V 40/20

(57) ABSTRACT

Methods of generating a mobility assessment for a subject are provided. Aspects of the methods include: instructing the subject to perform an activity including an oscillatory motion; generating a visual recording of the subject performing the activity using a recording device; extracting time series data from the visual recording using a dynamic algorithm; generating one or more musculoskeletal movement biomarkers from the time series data; and producing the mobility assessment for the subject from the one or more musculoskeletal movement biomarkers. Also provided are systems for use in practicing methods of the invention.

### CROSS REFERENCE TO APPLICATIONS

This application claims the benefit of U.S. Provisional Application Ser. No. 63/303,865, filed on Jan. 27, 2022, which application is incorporated by reference herein.

### STATEMENT REGARDING FEDERALLY SPONSORED RESEARCH

This invention was made with government support under grant no. R01 AR072669, awarded by The National Institutes of Health. The government has certain rights in the invention.

### INTRODUCTION

Chronic pain and musculoskeletal injuries are the most common cause of disability in the United States. Physical therapy (PT) is a first-line intervention for many musculoskeletal injuries, often showing superior clinical benefits as compared to surgery for conditions involving the back, knee, or shoulder. Although PT services have classically been delivered in clinic through face-to-face interactions between patients and providers, this in-person requirement disproportionately hinders the rehabilitation of underserved populations with less access to in-person care. For example, the elderly, a population that is most in need of PT services, faces the greatest demographic and socioeconomic barriers hindering their ability to seek in-person care and effecting significant deficits in their quality-of-life. One method to improve healthcare access within underserved communities is a technological framework for closed-loop, computerized tele-PT. However, such a design requires the ability to remotely diagnose musculoskeletal pathology. Unfortunately, remote diagnosis of musculoskeletal pathology, apart from often requiring the supervision of a medical professional or trained technician, relies on expensive marker- or sensor-based techniques.

### SUMMARY

The inventors discovered that recent advances in machine learning allow for the creation of a quick and affordable diagnostic model for assessing musculoskeletal pathology using the automated assessment of musculoskeletal system movements. Methods of generating a mobility assessment for a subject are provided. Aspects of the methods include: instructing the subject to perform an activity including an oscillatory motion; generating a visual recording of the subject performing the activity using a recording device; extracting time series data from the visual recording using a dynamic algorithm; generating one or more musculoskeletal movement biomarkers from the time series data; and producing the mobility assessment for the subject from the one or more musculoskeletal movement biomarkers. Also provided are systems for use in practicing methods of the invention.

### BRIEF DESCRIPTION OF THE FIGURES

FIG. 1 provides a depiction of a method for generating a mobility assessment in accordance with an embodiment of the invention.

FIG. 2 provides a flow diagram depicting a method of instructing the subject in accordance with an embodiment of the invention.

FIGS. 3A to 3D provide a depiction of a workflow of an experiment performed in accordance with an embodiment of invention. (A) a 3D printed string holder placed over a behavior box. (B) a timeline of mouse surgery and string-pulling training. (C) a depiction of neural network training. (D) a depiction of waveform filtering.

FIGS. 4A to 4F provide results of an experiment performed in accordance with an embodiment of invention. (A) a depiction of the overlay of 10 cycles of string-pulling behavior for an example mouse prior to injury. (B) a Y-axis trace or waveform generated for reaching and pulling movements. (C) a correlation of left and right arm movements at baseline. (D) a Y-axis trace or waveform generated for reaching and pulling movements before and after injury. (E) a depiction of full width at half maximum (FWHM). (F) FWHM for repaired and non-repaired injuries over the course of four weeks.

FIGS. 5A to 5C provide results of an experiment performed in accordance with an embodiment of invention. (A) FWHM for injured and control arms over the course of four weeks. (B) velocity for injured and control arms over the course of four weeks. (C) acceleration for injured and control arms over the course of four weeks.

FIGS. 6A to 6F provide results of an experiment performed in accordance with an embodiment of invention. (A) four components of principle component analysis overlayed. (B) a depiction of bispectral coherence analysis. (C) a depiction of the cumulative variance explained by four principal components for a representative mouse during a pre-injury baseline. (D) a depiction of the cumulative variance explained by two principal components as it changes over the course of four weeks. (E) a stem plot of the eigenvector weights for PC1 for a representative animal at baseline. (F) a depiction of eigenvector weights for the left and right arm as they change over the course of four weeks.

FIGS. 7A to 7C provide results of an experiment performed in accordance with an embodiment of invention. (A) a depiction of the amplitude of reach and pull epochs for the left and right arm as they change over the course of four weeks. (B) a depiction of the time of reach and pull epochs for the left and right arm as they change over the course of four weeks. (C) a depiction of movement symmetry of reach and pull epochs between the left and right arm as it changes over the course of four weeks.

FIGS. 8A to 8H provide results of an experiment performed in accordance with an embodiment of invention. (A) a depiction of the overlay of multiple cycles of string-pulling behavior for a human subject. (B) a Y-axis trace or waveform generated for reaching and pulling movements. (C) a depiction of FWHM for injured, uninjured, and control shoulders. (D) a depiction of velocity and acceleration for injured, uninjured, and control shoulders. (E) a depiction of PC1 eigenvector magnitude for injured, uninjured, and control shoulders. (F) a depiction of the amplitude of reach and pull epochs for injured, uninjured, and control shoulders. (G) a depiction of the time of reach and pull epochs for injured, uninjured, and control shoulders. (H) a depiction of the movement dynamic range ratio of the hand to the elbow for injured, uninjured, and control shoulders.

### DETAILED DESCRIPTION

Methods of generating a mobility assessment for a subject are provided. Aspects of the methods include: instructing the subject to perform an activity including an oscillatory motion; generating a visual recording of the subject performing the activity using a recording device; extracting time series data from the visual recording using a dynamic algorithm; generating one or more musculoskeletal movement biomarkers from the time series data; and producing the mobility assessment for the subject from the one or more musculoskeletal movement biomarkers. Also provided are systems for use in practicing methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, methods of generating a mobility assessment for a subject are provided. Aspects of the methods include: instructing the subject to perform an activity including an oscillatory motion; generating a visual recording of the subject performing the activity using a recording device; extracting time series data from the visual recording using a dynamic algorithm; generating one or more musculoskeletal (MSK) movement biomarkers from the time series data; and producing the mobility assessment for the subject from the one or more MSK movement biomarkers. Also provided are systems for use in practicing methods of the invention.

### Instructing the Subject/Activities Including Oscillatory Motion

Instructions that may be communicated to the subject may vary and include, but are not limited to, those found below. In some instances, the one or more instructions include, but are not limited to, instructions guiding the subject through calibrating or adjusting one or more factors or settings affecting the quality of a visual recording, instructions guiding the subject through performing an activity including an oscillatory motion, instructions notifying the subject when to begin performing the activity, instructions notifying the subject when to cease or end performing the activity, etc. In some embodiments, the one or more instructions may be communicated to the subject through any number of various visual or audio means including, but not limited to, those found below.

As described above, embodiments of the methods may include instructing the subject through calibrating or adjusting one or more factors or settings affecting the quality of a visual recording. In some embodiments, the one or more factors or settings are calibrated or adjusted such that the generated visual recording is of a sufficient quality. By sufficient quality it is meant the visual recording is capable of being used to produce time series data from which accurate MSK movement biomarkers may be generated as is described in greater detail below. In other words, a dynamic algorithm is capable of extracting accurate time series data from a visual recording of sufficient quality. In some embodiments, the one or more factors or settings affecting visual recording quality may include a lighting setting. In these cases, the subject may be informed that the environment the recording device is presently arranged or configured to generate a visual recording of is too dark or too bright to generate a visual recording of sufficient quality. The subject may then be instructed to adjust a physical configuration of the recording device or a physical factor effecting the amount of light in the relevant environment (e.g., a window shade or light switch) in order to ensure the visual recording device is capable of generating a visual recording of sufficient quality. In some embodiments, the one or more factors or settings affecting visual recording quality may include one or more settings of the recording device. The one or more settings of the recording device may include, but are not limited to, the frame rate, shutter speed, recorded resolution, ISO settings, white balance, aperture settings, picture style, etc. of the recording device. The subject may be instructed to adjust one or more settings of the recording device (e.g., physically, or electronically) in order to ensure the visual recording device is capable of generating a visual recording of sufficient quality. In some cases, the one or more recording device settings may be automatically adjusted using, e.g., computer code or a computer program, to ensure the visual recording device is capable of generating a visual recording of sufficient quality.

In some embodiments, the one or more factors or settings affecting the quality of a visual recording includes the distance between the subject and the recording device. In these cases, the instructions may position the subject a distance from the recording device. For example, the subject may be instructed to move further away from or closer to the recording device based on automatically generated feedback from the recording device. In some embodiments, the feedback may be automatically generated from visual images generated by the recording device in real time. In some embodiments, the visual images may include one or more body landmarks of the subject. The one or more body landmarks may include, but are not limited to, the subjects hands, hips, knees, ankles, feet, elbows, shoulders, scapula, neck, chest, or facial features. For example, the feedback may be generated based on the resolution of the recording device and the number of pixels between the eyes of the subject in a visual image generated by the recording device, e.g., in real time. In some cases, the one or more body landmarks are selected based on a body part from which time series data and/or one or more MSK movement biomarkers will be generated as described in greater detail below. In some embodiments, the subject may wear or be affixed to a motion tracking marker or sensor. The motion tracking marker or sensor may include a visual pattern or may emit a detectable data signal. In these cases, the positioning feedback may be generated using the motion tracking marker or sensor. For example, the feedback may be automatically generated based on the resolution of the recording device and the number of pixels between components of the visual pattern of the marker or sensor in a visual image generated by the recording device in real time.

As described above, embodiments of the methods may include instructing the subject to move further away from or closer to the recording device based on automatically generated feedback from the recording device. In some embodiments, the recording device may emit a laser beam such as, e.g., an infrared (IR) laser beam, a near infrared (NIR) laser beam, or a laser beam of visible light. The laser can be emitted using any capable diode such as, e.g., a vertical-cavity surface-emitting laser (VCSEL) diode. In these instances, the recording device may include radar, sonar, or a Light Detection and Ranging (LiDAR) scanner. In some embodiments, the positioning feedback may be generated from the emitted laser beam of the recording device (e.g., the VCSEL beam). For example, the instructions may prompt the subject to move further away from or closer to the recording device based on automatically generated feedback from a LiDAR scanner of the recording device until, e.g., the subject is positioned a distance from the recording device that allows for the recording device to generate a visual recording of sufficient quality.

Embodiments of the methods may include instructing the subject to perform an activity including an oscillatory motion. By oscillatory motion is meant a motion that repeats itself in time or, i.e., is periodic. Activities including an oscillatory motion, in accordance with embodiments of the methods, may vary and include, but are not limited to, those found below. In some embodiments, the oscillatory motion may include the movement of one or more body parts of the subject. The one or more body parts may include, but are not limited to, the subject's arms, legs, hands, pelvis, hips, back, thorax, neck, ankles, feet, hands, phalanges, or shoulders. In some embodiments, the one or more body parts includes a joint of the subject such as, e.g., a ball and socket joint, saddle joint, hinge joint, condyloid joint, pivot joint, or gliding joint. In embodiments where the one or more body parts includes a ball and socket joint, the ball and socket joint may be one or both of the subject's shoulder or hip joints. In embodiments where the one or more body parts includes a hinge joint, the hinge joint may be one or both of the subject's elbow, knee, or ankle joints or one or more of the subject's interphalangeal joints. In embodiments where the one or more body parts includes a condyloid joint, the condyloid joint may be one or both of the subject's radiocarpal joints. In cases where the one or more body parts includes a joint, the oscillatory motion may include the movement of one or more tendons or muscles associated with the joint. For example, in embodiments where the oscillatory motion includes the movement of the subject's shoulder joints, the oscillatory motion may further include the movement of the subject's rotator cuffs.

As described above, the oscillatory motion may include the movement of one or more body parts of the subject. The movement of the one or more body parts may vary and includes, but is not limited to, a repeated abduction, adduction, flexion, extension, or circumduction of the one or more body parts. For example, the oscillatory motion may include the repeated abduction and adduction of one or both of the subject's hip joints. The movement of the one or more body parts may also include open or closed kinetic chain movements. In some embodiments, the movement includes an open kinetic chain movement. For example, the movement might be side-lying leg raises. In some embodiments, the movement includes a closed kinetic chain movement. For example, the movement might be lateral lunges. In some embodiments, resistance may be added or applied to the movement of the one or more body parts. By resistance is meant a force counteracting the movement of the one or more body parts. For example, in embodiments where the movement is lateral lunges, a weight or a resistance band may be, e.g., affixed to one or both ankles of the subject. In other embodiments, no resistance is added or applied to the movement of the one or more body parts.

In some embodiments, the movement of the one or more body parts of the subject may be guided by a device or machine. In some cases, the device or machine might apply resistance. In other cases, the device or machine might apply no resistance or minimal resistance. In some embodiments, the device or machine might include a sensor generating information regarding the movement of the one or more body parts. For example, the device or machine may include a string or rope configured to be vertically pulled, and a sensor may measure the rate at which the string or rope is vertically pulled (e.g., the meters of rope pulled per second). In some embodiments, the device or machine might include a motion tracking marker that allows the position of one or more components of the device or machine to be easily determined from a generated visual recording. For example, in embodiments where the device or machine includes a string or rope configured to be vertically pulled, a motion tracking marker may be affixed to the string or rope. In other embodiments, the device or machine does not include a sensor or a motion tracking marker.

As discussed above, embodiments of the methods may include instructing the subject to perform an activity including an oscillatory motion. In some embodiments, the oscillatory motion is chosen or selected based on one or more body parts of interest. In some embodiments, the one or more body parts of interest have experienced an injury or were targeted in a surgery. In some embodiments, the one or more body parts of interest have been targeted by physical therapy (PT). In some embodiments, the one or more body parts of interest are essential to the completion of a task such as, e.g., a task associated with the employment of the subject or a hobby of the subject. In these embodiments, the oscillatory motion may also be chosen or selected based on the task. In some embodiments, the one or more body parts of interest are affected by a disease or condition of interest. The disease or condition of interest may vary and includes, but is not limited to, arthritis, tendonitis, a tendon or myotendinous tear, a hernia, old age, chronic health problems associated with bad posture, etc. In these embodiments, the oscillatory motion may also be chosen or selected based on the disease or condition of interest. In some embodiments, there is evidence the subject has the disease or condition. In some embodiments, the subject has been diagnosed with the disease or condition. In some embodiments, the oscillatory motion may be chosen based, at least in part, on the devices or machines available or accessible to the subject as discussed above.

In some embodiments, the one or more body parts of interest include one or both of the subject's knees. In these cases, the oscillatory motion may include the repeated flexion and extension of one or both of the subject's knee joints. For example, the selected activity including the oscillatory motion may be forward lunges, reverse lunges, side lunges, or deadlift.

In some embodiments, the one or more body parts of interest include one or both of the subject's hips. In these cases, the oscillatory motion may include the repeated flexion, extension, abduction, adduction, or circumduction of one or both of the subject's hip joints. For example, the selected activity including the oscillatory motion may be forward lunges, reverse lunges, side lunges, deadlift, crab walk, monster walk, leg circles, or hip circles.

In some embodiments, the one or more body parts of interest include one or both of the subject's ankles. In these cases, the oscillatory motion may include the repeated flexion, extension, abduction, adduction, or circumduction of one or both of the subject's ankle joints. For example, the selected activity including the oscillatory motion may be heel raises or ankle circles.

In some embodiments, the one or more body parts of interest includes the subject's back. In these cases, the oscillatory motion may include the repeated flexion, extension, abduction, adduction, or circumduction of the subject's back. For example, the selected activity including the oscillatory motion may be back extensions, side bending, forward bending, squats, or hip circles.

In some embodiments, the one or more body parts of interest include one or both of the subject's hands. In these cases, the oscillatory motion may include the repeated flexion, extension, abduction, adduction, or circumduction of one or both of the subject's radiocarpal joints. For example, the selected activity including the oscillatory motion may be hand circles, wrist rolls, or the repeated writing of a symbol or shape.

In some embodiments, the one or more body parts of interest include one or both of the subject's shoulders. In these cases, the oscillatory motion may include the repeated flexion, extension, abduction, adduction, or circumduction of one or both of the subject's shoulder joints. For example, the selected activity including the oscillatory motion may be vertical rope pulls, arm circles, side raises, front raises, or shoulder press.

As discussed above, embodiments of the methods may include instructing the subject to perform an activity including an oscillatory motion. In some embodiments, the oscillatory motion is repeated two or more times, such as five or more times, or ten or more times, or twenty or more times, or fifty or more times, or one hundred or more times. In some embodiments, the oscillatory motion is repeated for a set amount of time. For example, the oscillatory motion may be repeated for five seconds or more, such as ten seconds or more, or twenty seconds or more, or thirty seconds or more, or sixty seconds or more, or one hundred seconds or more. In some embodiments, the oscillatory motion is repeated a number of times sufficient to accurately generate one or more MSK movement biomarkers. By accurately generate is meant the one or more MSK movement biomarkers are not affected or are minimally affected by noise and/or meet a standard of statistical relevance (e.g., as determined by a statistical test such as a T-test or an ANOVA test).

Embodiments of the methods may include instructions notifying the subject when to begin or cease (i.e., end/stop) performing the activity including an oscillatory motion. The notification to begin performing the activity may occur after the subject has received instructions guiding the subject through performing the activity (e.g., what the activity is or how to perform the activity) as is discussed above. In some embodiments, the notification to begin performing the activity may occur based on a determination regarding if the visual recording device is presently configured to generate a visual recording of sufficient quality. For example, the notification to begin may occur after the subject has received instructions guiding them through calibrating or adjusting one or more factors or settings affecting the quality of the visual recording as is discussed above. The notification to cease or stop performing the activity may be based on a number of repeated oscillatory motions for which a visual recording has been generated. In some embodiments, the notification to cease or stop may occur after a visual recording has been generated for a number of repeated oscillatory motions determined to be sufficient to accurately generate one or more MSK movement biomarkers. In these instances, it may be automatically determined when a number of repeated oscillatory motions sufficient to accurately generate one or more MSK movement biomarkers has been visually recorded. In these cases, the number of repeated oscillatory motions determined to be sufficient may vary, e.g., based on the oscillatory motions recorded and/or the quality of the visual recording.

As described above, embodiments of the methods may include instructing the subject to perform one or more tasks or activities. The instructions may be communicated to the subject through any number of various visual or audio means including, but not limited to, a display device providing visual information and/or a loudspeaker. The display device may be an electronic display device such as, e.g., a liquid crystal display (LCD), an organic light-emitting diode (OLED) display or an active-matrix organic light-emitting diode (AMOLED) display. In some embodiments, the display device may include a projector. In some embodiments, the instructions may be communicated to the subject through written text, audible speech, images, or videos. In embodiments where the communication includes written text, one or more aspects of the text such as, e.g., the language, size, or font of the text or the speed at which the text is displayed may vary based on the preferences of the subject. In embodiments where the communication includes audible speech, one or more aspects of the speech such as, e.g., the language, accent, volume, or speed of the text may vary based on the preferences of the subject. In some embodiments, one or more images or videos are provided to the subject instructing the subject on how to perform the activity including the oscillatory motion as described above, e.g., on a step-by-step basis. In some cases, the subject may be given the option to skip the step-by-step instructions if they are familiar with the activity. In some embodiments, the level of detail of the instructions may vary depending on the preferences of the subject. The instructions provided to the subject, in accordance with embodiments of the methods, are sufficient (e.g., contain a sufficient level of detail) for the subject to understand the one or more tasks or activities they are prompted to perform as discussed above.

### Generating the Visual Recording

Embodiments of the methods include generating a visual recording of the subject performing the activity (e.g., including an oscillatory motion as described above) using a recording device. The recording device may include any device capable of generating a sequence of visual images over time. In some embodiments, the recording device may include, but is not limited to, digital cameras or camcorders. In some embodiments, the recording device may be a smartphone camera or a computer camera (e.g., a webcam). For example, the recording device may be an iPhone camera, an Android camera, a personal computer (PC) camera such as, e.g., a tablet computer camera, a laptop camera (e.g., a MacBook or an XPS laptop camera), etc. In embodiments of the methods, the recording device is capable of generating a visual recording of sufficient quality. For example, the recording device may be capable of generating a visual recording having at least a minimum number of frames per second (FPS) or a minimum resolution. The minimum FPS or minimum resolution may vary, e.g., depending on the size of the subject or the oscillatory motion being performed by the subject. In some embodiments, the recording device is capable of producing a video having fifteen FPS or more, such as thirty FPS or more, or sixty FPS or more, or two hundred forty FPS or more, or five hundred FPS or more, or one thousand FPS or more, or fifteen thousand FPS or more. In some embodiments, the recording device is capable of producing a video having a resolution of 360p or more, such as 720p or more, or 1080p or more, or 2160p or more, or 4000p or more, or 4320p or more, or 8640p or more. In some embodiments, the recording device is capable of generating an audio recording (e.g., the recording device includes a microphone).

In some embodiments, the visual recording may be generated by placing or setting the recording device on a stable surface. For example, the recording device may be placed on the floor, on a desk or table, on a tripod, on workout equipment at a gym or in a clinic, etc. In some embodiments, the visual recording may be generated while the recording device is held by a human such as, e.g., the subject or an agent of the subject. In embodiments where the recording device is held by the subject, the subject may record themselves performing the oscillatory motion in a reflective surface such as a mirror. In some embodiments, the recording device may include a stabilizer. In some embodiments, the visual recording may be stabilized using, e.g., computer code or a computer program/algorithm after it has been generated using the recording device. The visual recording may be generated in any environment where the subject can perform the activity including an oscillatory motion as described above. For example, the recording may be generated at the subject's home, at the subject's place of work, at a clinic or hospital, outside, in a gym or workout facility, etc. In embodiments where the oscillatory motion includes the use of a device or machine (e.g., as discussed above), the recording may be generated in any location the subject has access to the device or machine such as, e.g., the subject's home, a clinic or hospital, or in a gym or workout facility.

In some embodiments, the visual recording may be generated without the use of a motion tracking marker or sensor. In some embodiments, the visual recording may include the use of a motion tracking marker or sensor. In these embodiments, the motion tracking marker or sensor may vary and includes, but is not limited to, a wearable device such as a smartwatch (e.g., Apple watches, Garmin watches, or Fitbit® watches). In some embodiments, the wearable device may include motion sensors (e.g., accelerometers, gyroscopes, and magnetometers), electrical sensors (e.g., electrocardiogram sensors), or light sensors (e.g., photoplethysmography (PPG) sensors). The motion tracking marker or sensor may be worn by or affixed to the subject such as, e.g., a body part of subject performing the oscillatory motion as described above. In some embodiments, the motion tracking marker or sensor includes a visual pattern or emits an audio frequency. For example, a smartwatch may be configured to display a striped pattern and/or emit a rapid series of audio chirps. The visual pattern may be used to determine a distance the motion tracking marker or sensor is from the recording device or a distance the motion tracking marker or sensor has traveled between two sequentially generated visual images using, e.g., the resolution of the recording device and the number of pixels between components of the visual pattern. The audio frequency may be used to determine a velocity the motion tracking marker or sensor is traveling away from or towards the recording device using, e.g., a change in the audio frequency (Doppler effect) or a change in the amount of time between audio chirps.

As discussed above, embodiments of the methods may include a motion tracking marker or sensor affixed to the subject, a marker or sensor affixed to a device or machine used in performing the oscillatory motion, or an audio recording device. In embodiments where additional measurements or recordings are generated apart from the visual recording, the methods may further include associating or aligning the additional measurements or recordings with the visual recording. For example, the one or more additional measurements may be timestamped using a timeline generated from the visual recording such that the additional measurements/recordings and the visual recording have a common time reference or exist on a single timeline. Embodiments of the methods may further include transmitting the visual recording and the one or more additional measurements, e.g., to a computer or mobile device application, a website, a processor, etc. in order for time series data to be extracted from the visual recording and the one or more additional measurements, as discussed in greater detail below. In some embodiments, the recording device and the one or more additional sensors transmit data directly to a processor including a program or application configured to extract time series data as discussed in greater detail below.

### Extracting Time Series Data

Embodiments of the methods include extracting time series data from the visual recording (e.g., as described above) using a dynamic algorithm. By time series (i.e., timestamped) data is meant a series of data points indexed in time order. In some embodiments, the time series data includes the location or position of one or more body parts of the subject. For example, the time series data may include the location or position of a body part performing the oscillatory motion as described above and the time at which the body part was at the location (i.e., located at the position). In some embodiments, the time series data includes a waveform generated by graphing the location of a body part of the subject on an axis over time. For example, the horizontal location or position (e.g., coordinates) of a body part may be graphed versus time such that a sinusoidal waveform may be generated. The location of a body part of the subject on the horizontal, vertical, or depth axis may be extracted in order to, e.g., generate a waveform of the location of a body part of the subject on an axis over time as described above.

As discussed above, embodiments of the methods include extracting time series data from the visual recording using a dynamic algorithm. By dynamic algorithm is meant an algorithm configured to change based on data processed by (e.g., passed into or received by) the algorithm. In some embodiments, the dynamic algorithm is a machine learning algorithm, such as a machine learning algorithm that uses an artificial neural network. For example, the artificial neural network may be a recurrent neural network (RNN), convolutional neural network (CNN), or region-convolutional neural network (R-CNN). The dynamic algorithm may include any algorithm capable of identifying one or more body parts of the subject from a visual image. In some embodiments, the algorithm includes a deep learning algorithm such as, e.g., a ResNet, InceptionNet, VGGNet, GoogLeNet, AlexNet, EfficientNet, or YOLONet neural network. In embodiments where the algorithm includes a deep learning algorithm (e.g., an artificial neural network) the algorithm may be three or more layers deep, such as five or more layers deep, or ten or more, or twenty or more, or fifty or more, or one hundred or more.

The dynamic algorithm may be trained using any relevant data set or, e.g., any data set that includes visual images labeled with one or more relevant body parts. For example, the dynamic algorithm may be trained, at least in part, using DeepLabCut™, DeepPoseKit, LEAP, SLEAP, or Anipose. In some embodiments, a human (e.g., the subject or a technician) may label one or more images or video frames of the visual recording with one or more relevant body parts. The manually labeled images or video frames of the visual recording may then be used to train the dynamic algorithm. In embodiments where a human labels one or more images of the visual recording, the images may be outliers. In some embodiments, outlier images are images with a minimum Euclidean distance between two successively labeled points (i.e., images where the one or more body parts jumps a minimum distance between two successive images or video frames). For example, outlier images may include images where a body part jumps twenty or more pixels between two successive images or video frames. In some embodiments, the dynamic algorithm does not require any additional training after initially receiving or processing the visual recording of the subject performing the oscillatory motion as discussed above.

As discussed above, embodiments of the methods include extracting time series data from the visual recording (e.g., as described above) using a dynamic algorithm. In some embodiments, the extracted time series data (e.g., a waveform) is processed or cleaned before being used to generate one or more MSK movement biomarkers as discussed in greater detail below. The extracted time series data may be processed or cleaned in order to, e.g., reduce noise, minimize distortion, better capture the minima and maxima of a waveform, smooth a waveform, or increase the accuracy or precision of one or more MSK movement biomarkers generated from the extracted time series data. In some embodiments, the extracted time series data may be processed or cleaned using a filter such as, e.g., a signal processing filter. The filter may be a high pass filter, a low pass filter, a band pass filter, or a notch filter. In some embodiments, the filter may be a linear continuous-time filter including, but not limited to, a Butterworth filter, Chebyshev filter, Savitzky-Golay filter, elliptic (Cauer) filter, Bessel filter, Gaussian filter, Optimum “L” (Legendre) filter, or Linkwitz-Riley filter. In embodiments where it is desired to have a flat frequency response in the passband, a Butterworth filter may be used. For example, in some embodiments a 1st order Butterworth high pass filter followed by a 7th order Butterworth low pass filter may be used in succession in order process or clean the time series data before it is used to generate one or more MSK movement biomarkers as discussed in greater detail below.

### Musculoskeletal (MSK) Movement Biomarkers

Embodiments of the methods include generating one or more MSK movement biomarkers from the time series data generated as discussed above. By MSK movement biomarker is meant a measurable indicator of a state or condition of one or more components of the musculoskeletal system generated from the movement of one or more body parts of the subject as discussed above. MSK movement biomarkers, in accordance with embodiments of the methods, may vary and include, but are not limited to, those found below.

In some embodiments, the one or more MSK movement biomarkers may be indicative of the range of motion, strength, flexibility, or neuromuscular control of one or more components or body parts of the musculoskeletal system. For example, the full width at half maximum (FWHM) for waveform peaks (i.e., capturing the transition between changes in the direction of an oscillatory motion) may be generated as an indicator of neuromuscular control. In some embodiments, the one or more MSK movement biomarkers includes MSK movement biomarkers thereof selected from the group consisting of oscillatory motion amplitude, duration, FWHM, acceleration, and velocity.

In some embodiments, one or more MSK movement biomarkers may be generated using principal component analysis. In these instances, one or more of the MSK movement biomarkers may be generated by comparing different principal components of a principal component analysis. For example, one or more of the MSK movement biomarkers may be generated by comparing different principal components of a principal component analysis using, e.g., bispectral coherence. In some embodiments, one or more MSK movement biomarkers may be generated by comparing the movement of a body part on two more different axes as the oscillatory motion is performed. In some embodiments, one or more MSK movement biomarkers may be generated by comparing the movement of separate body parts performing the oscillatory motion (e.g., on the same or different axes). For example, one or more MSK movement biomarkers may be generated based on or related to the correlation or the relationship between separate body parts performing the oscillatory motion. In some embodiments, the separate body parts may perform the oscillatory motion at separate times or may be performing a separate phase of the oscillatory motion at any one time. In other embodiments, the separate body parts may perform the oscillatory motion at the same time and may be performing the same phase of the oscillatory motion at any one time. In some embodiments, the separate body parts are equivalent bilaterally symmetric body parts (i.e., a left knee and a right knee or a left hand and a right hand). In some embodiments, the separate body parts are bilaterally asymmetric to each other.

In some embodiments, one or more of the MSK movement biomarkers may be related to the symmetry of the oscillatory motion. For example, one or more of the MSK movement biomarkers may be generated by comparing the peaks and troughs of a generated waveform or the rising and falling components of a generated waveform (e.g., the reach and pull epochs, or flexion and extension epochs of some oscillatory motions). In some embodiments, the symmetry is between abduction and adduction movements or flexion and extension movements. In some embodiments, the symmetry is between arclengths of circumduction movements. In some embodiments, the symmetry is between reaching and pulling movements.

In some embodiments, one or more of the MSK movement biomarkers may be generated based on the angle between a body part and a reference plane (e.g., the ground) or the angle between two body parts. For example, MSK movement biomarkers may be generated based on the angle between a subject's forearm and upper arm. In some embodiments, one or more of the MSK movement biomarkers may be generated based on the movement dynamic range of one or more body parts. For example, one or more of the MSK movement biomarkers may be a movement dynamic range ratio between two body parts such as, e.g., the movement dynamic range ratio between a hand and elbow of the subject.

In some embodiments, one or more of the MSK movement biomarkers may be generated using optical flow algorithms such as, e.g., optical flow deep learning algorithms. Optical flow algorithms may monitor changes in pixel intensities across time in order to, e.g., estimate or determine a metric related to the motion of an object (e.g., a body part of the subject) captured in the visual recording. In some embodiments, the velocity of a body part performing the oscillatory motion may be determined from the generated time series data using an optical flow algorithm. In some instances, the time series data may be extracted from the visual recording at least in part using an optical flow algorithm. In these cases, one or more of the MSK movement biomarkers may then be generated from the extracted time series data as discussed above.

In some embodiments, a visual recording may be generated (and e.g., time series data may be extracted) at two or more timepoints to generate two or more of the same MSK movement biomarkers. In some instances a visual recording may be generated at three or more timepoints to generate three or more of the same MSK movement biomarkers, such as four or more, or five or more, or ten or more. The two or more timepoints may be at least 30 seconds apart from each other, such as at least a day apart from each other, or at least a week apart from each other, or at least a month apart from each other, or at least a year apart from each other. In some instances, a first timepoint of the two or more timepoints may occur after an injury of the subject. In other instances, a first timepoint of the two or more timepoints may occur before an injury of the subject in order to, e.g., function as a baseline. In these instances, a subsequent timepoint may occur after an injury of the subject. In some instances, a first timepoint of the two or more timepoints may occur after the subject has received a medical intervention. In other instances, a first timepoint of the two or more timepoints may occur before the subject has received a medical intervention in order to, e.g., function as a baseline. In these instances, a subsequent timepoint may occur after the subject has received a medical intervention. In some embodiments, two or more of the same MSK movement biomarkers generated at different timepoints are used to determine a level of recovery of the subject after an injury or a surgery. In some embodiments, two or more of the same MSK movement biomarkers generated at different timepoints are used to determine a level of effectiveness of a medical intervention (e.g., physical therapy). In some embodiments, two or more of the same MSK movement biomarkers generated at different timepoints are used to determine a decline in the mobility of the subject. A visual recording may be generated (i.e., a timepoint may occur) every set number of minutes, hours, days or months while the subject is receiving a certain medical treatment or working a certain profession.

In some instances, the one or more MSK movement biomarkers generated from a visual recording may be is associated with an identifier of the subject. The identifier of the subject may vary, where examples of identifiers include, but are not limited to alpha/numeric identifiers (e.g., an identification number or a string of letters and/or numbers), codes such as, e.g., QR codes, barcodes, facial recognition metrics, etc. In some embodiments, the identifier may identify the subject through association with identifying information of the subject such as, but not limited to, the subject's full legal name, contact information, home address, social security number, a body landmark of the subject as discussed above such as, e.g., a facial feature, etc. In these embodiments, the association may occur in a database or in a datasheet (e.g., wherein the identifying information may be found by searching for the identifier). In these cases, it may be relatively difficult or impossible to associate the identifying information of the subject with the identifier without access to the database or the datasheet (i.e., the database or datasheet is secured and/or protected). In some instances the one or more MSK movement biomarkers and/or associated identifier may be saved via local storage and/or cloud storage and, e.g., may be saved to a database such as a data warehouse.

In some embodiments, correlations and relationships between health outcomes and one or more MSK movement biomarkers may be determined from previously saved MSK movement biomarkers such as, e.g., the MSK movement biomarkers saved to a data warehouse as discussed above. In some embodiments, correlations and relationships between the diagnosis of a disease or condition and one or more MSK movement biomarkers may be determined from previously saved MSK movement biomarkers. In some embodiments, correlations and relationships between the fitness of a subject for performing a task and one or more MSK movement biomarkers may be determined from previously saved MSK movement biomarkers. The previously saved MSK movement biomarkers may include MSK movement biomarkers generated from the subject presently obtaining the one or more MSK movement biomarkers and/or MSK movement biomarkers generated from other subjects for which one or more MSK movement biomarkers were previously obtained.

As discussed above, correlations and relationships between health outcomes, the diagnosis of a disease or condition, or the fitness of a subject for performing a task and one or more MSK movement biomarkers may be determined from previously saved MSK movement biomarkers such as, e.g., the MSK movement biomarkers saved to a data warehouse as discussed above. In some embodiments, the correlations and relationships may be determined, at least in part, using linear mixed-effects (LME) models. In some embodiments, the correlations and relationships may be determined, at least in part, using a package including statistical analysis functions such as, e.g., statsmodels. In some embodiments, the relationship or correlation may be determined, at least in part, using a dynamic algorithm. The dynamic algorithm may be a machine learning algorithm, such as, e.g., a machine learning algorithm using a neural network. In some embodiments, the neural network is a deep learning algorithm that is three or more layers deep, such as five or more layers deep, or ten or more, or twenty or more, or fifty or more, or one hundred or more. In embodiments where the dynamic algorithm is a machine learning algorithm, the machine learning algorithm may include, but is not limited to, a linear and/or logistic regression algorithm, a linear discriminant analysis algorithm, a support vector machine (SVM) algorithm, a random forest algorithm, or an XGBoost algorithm.

### The Mobility Assessment

Embodiments of the methods include producing a mobility assessment for the subject from one or more generated musculoskeletal movement biomarkers. The mobility assessment is a qualitative or quantitative determination regarding one or more mobility related matters pertaining to the subject. The mobility assessment, generated in accordance with embodiments of the methods, may vary and includes, but is not limited to, the components found below.

In some embodiments, the subject obtaining the mobility assessment may be human. In some embodiments, the subject has been diagnosed with a disease or condition, or there is evidence the subject has, or is at risk of developing, the disease or condition. The disease or condition may vary and includes, but is not limited to, arthritis, tendonitis, a tendon or myotendinous tear, a hernia, old age, chronic health problems such as, e.g., chronic pain or chronic problems associated with bad posture, etc. In some cases, the subject is sixty years of age or older. In other cases, the subject is younger than sixty years of age. In some instances, the subject has experienced an injury such as, e.g., an MSK injury. In these instances, the MSK injury may be an injury of the shoulder (e.g., a rotator cuff), a muscle strain or a muscle tear, or a sprain. The injury may have occurred at any point in time such as, e.g., longer than a year in the past or more recently than a year in the past. In some cases, the subject has received surgery such as, e.g., surgery on the back, knee, hip, ankle, or shoulder. The surgery may have occurred at any point in time such as, e.g., longer than a year in the past or more recently than a year in the past. In some instances, the subject's employment or a hobby of the subject may put the subject at an elevated risk of MSK injury. In some cases, the subject may regularly perform physical training exercises such as, e.g., strength, flexibility, or endurance training exercises.

In some instances, the mobility assessment includes a qualitative or quantitative determination regarding one or more mobility related matters pertaining to the subject relative to a baseline. The baseline may vary, and in some instances includes a cohort average value, such as an average level or value of a given MSK movement biomarker generated from a population or cohort of interest. By population or cohort is meant a group of people banded together or treated as a group, such as the categories of professionals, e.g., fire fighters or professional athletes, a group of people living in a specified locality, a group of people in the same age range, etc. In some instances, the baseline includes a prior value obtained from the subject, e.g., a value obtained from the subject 1 day prior to the generation of the most recent visual recording, or 1 week prior, or 1 month prior, or 6 months prior, or 1 year prior, or 5 years prior, etc. In such instances, the MSK movement biomarkers may indicate a temporal change of the one or more mobility related matters pertaining to the subject.

In some embodiments, the mobility assessment includes an interpretation of the one or more MSK movement biomarkers. For example, a relationship or correlation between one or more MSK movement biomarkers and a disease or condition may be determined. The correlation or relationship can be determined by comparing one or more MSK movement biomarkers generated from healthy patients with one or more MSK movement biomarkers generated from patients diagnosed with a disease or condition. In some cases, the correlation or relationship may be generated using a dynamic algorithm, such as a machine learning algorithm (e.g., a neural network). In these embodiments, the interpretation may include the likelihood that the subject has a disease or condition (e.g., a potential diagnosis). In these instances, the interpretation may include the severity or stage of the disease or condition. In some embodiments, the interpretation may include the likelihood or risk level the subject may have of developing a disease or condition.

In some embodiments, the interpretation may include a general assessment of the subject's MSK health or the health or condition of a specific component or body part of the subject's MSK system. For example, the interpretation may include a general assessment of the subject's shoulder joint condition (e.g., shoulder mobility is overall good, somewhat poor, overall poor, etc.). In some embodiments, the interpretation may include a general assessment of a specific movement performed by the subject (e.g., the quality of the movement). In these embodiments, the interpretation may include a determination regarding whether one or more body parts is compensating for another body part or whether one or more body parts is being compensated for. In some embodiments, the interpretation may include a general assessment of a subject's fitness for performing a task (e.g., a movement) or undertaking a duty or responsibility (e.g., associated with the subject's employment). By fitness is meant the ability of the subject to perform and/or the risks associated with the subject undertaking a task or tasks associated with the duty or responsibility. For example, the interpretation may include a general assessment regarding the fitness of a sports professional or recreational athlete for returning to practice.

In some embodiments, the mobility assessment may include a suggested next course of action. In embodiments where a next course of action is suggested, the suggested course of action may vary. In some instances, the course of action includes obtaining additional tests or consulting with additional medical professionals. For example, the suggested course of action may include consulting a specialist wherein a secondary opinion may be obtained, or additional testing may be recommended or ordered. In some embodiments, the suggested course of action may include a temporary or permanent modification to the subject's responsibilities of employment. For example, the suggested course of action may include a period of time wherein the subject should avoid performing a particular task or movement. In some embodiments, the suggested course of action may include an explanation regarding typical manners in which an individual may develop a higher risk of developing a disease or condition and steps the subject may take to avoid or mitigate the risk. For example, the suggested course of action may include preventative measures, such as, e.g., a recommended exercise routine or recommended braces (e.g., an ankle or knee brace). In some embodiments, the suggested course of action may include a potential treatment regimen or therapy recommendation. By treatment regimen is meant a treatment plan that specifies the quantity, the schedule, and the duration of treatment. For example, the treatment regimen may include a suggested physical therapy, or a suggested lifestyle change (e.g., dietary or exercise routines, etc.).

In some instances, the mobility assessment may include an evolution of MSK system condition, a disease or condition severity, or a specific movement quality. By evolution is meant a progression of a metric over time such as, e.g., the progression of MSK system condition, a condition or disease severity, or a specific movement quality over time. In some cases, the evolution is generated based at least in part on one or more previously obtained mobility assessments or MSK movement biomarkers. In some embodiments, the mobility evolution includes an explanation of how the relevant metric has changed over time. For example, the mobility evolution may include a peak, periods of decline or incline, and whether the metric is in a period of incline or decline at the time the present mobility assessment was obtained. In some embodiments, the mobility assessment may include an assessment of the effectiveness of a previously suggested next course of action (e.g., as described above). For example, the mobility assessment may include an assessment of the effectiveness of previously suggested physical therapy or exercise routines. The assessment of effectiveness may be obtained based on whether the mobility evolution indicates the level of a metric is in a period of incline or decline at the time the present mobility assessment was obtained.

In some embodiments, the mobility assessment may include one or more mobility scores. By mobility score is meant a quantitative evaluation of the subject's overall mobility, the mobility of one or more body parts of the subject's MSK system, a specific movement performed by the subject, or the subject's fitness for performing a task compared with a baseline. The baseline may vary, and in some instances includes the average of data associated with a cohort of interest. In some instances, the baseline includes prior data obtained for the subject. In embodiments where the one or mobility scores includes an evaluation of a specific movement performed by the subject, one or more of the scores may indicate whether one or more body parts of the subject is being compensated for or is compensating for another body part. The one or more mobility scores may be a composite of multiple MSK movement biomarkers, e.g., compared with a baseline.

In some instances, the mobility assessment may include one or more personalized insights. A personalized insight may vary and includes, but is not limited to, the detection of an anomaly, a classification, the detection of a cluster, or a forecast. In some instances, the personalized insight includes an insight regarding the subject individually. In other instances, the personalized insight includes an insight regarding a group or cohort in which the subject belongs. In embodiments where the insight includes the detection of an anomaly, the insight may include the identification of unusual data. For example, the insight may be that a specific movement performed by the subject is abnormal and what is abnormal about the movement (e.g., when compared to a baseline as described above). In embodiments where the insight includes a classification, the insight may include the identification of a group with similar data to the subject and, e.g., assigning and comparing the results and/or data of the subject to the group. For example, the insight may be that the subject has better shoulder mobility than 70% of people in their age group. In embodiments where the insight includes the detection of a cluster, the insight may include finding groups with similar results. For example, the insight may be that a profession or hobby has the highest rate of MSK injuries or the fastest decline in overall mobility.

As discussed above, the mobility assessment may include one or more personalized insights. In some embodiments, the personalized insight may include a forecast. In some embodiments, the forecast may include a predicted future outcome such as, e.g., a health or mobility outcome prediction for the subject. The health or mobility outcome can be predicted, at least in part, using a mobility assessment or MSK movement biomarker obtained as discussed above. For example, the predicted health or mobility outcome may be that the subject has a high risk of developing a specific disease or condition (e.g., arthritis or chronic pain). In some instances, the health or mobility outcome can be predicted at least in part using a dynamic algorithm. The dynamic algorithm may be a machine learning algorithm, such as a machine learning algorithm that uses an artificial neural network. In some instances, the mobility assessment is used to determine if a particular injury, surgery, or medical intervention has affected the subject's predicted health or mobility outcomes. In instances where the subject is recorded at two or more timepoints to generate two or more mobility assessments and/or MSK movement biomarkers, the two or more mobility assessments and/or MSK movement biomarkers may be used to, e.g., determine any changes in the subject's overall mobility, the mobility of one or more body parts of the subject's MSK system, the quality of a specific movement performed by the subject, or the subject's fitness for performing a task. In some cases, some combination of the two or more mobility assessments and/or MSK movement biomarkers is used to determine if the subject has experienced a decline in mobility.

In some embodiments, the mobility assessment may include notes or explanations aiding the subject, or a person associated with the subject, in interpreting the results of the mobility assessment. In some embodiments, the mobility assessment may include a background section such as, e.g., a background section explaining the purpose of the mobility assessment and the implication of certain results. In some embodiments, the mobility assessment may include visual means aiding the subject, or a person associated with the subject, in interpreting the findings of the mobility assessment (e.g., figures, charts, images, etc.). The visual means may be a component of, or accompany, any of the components the mobility assessment is comprised of such as, e.g., any of the components described above.

In some embodiments, the mobility assessment may be obtained or generated, at least in part, using a dynamic algorithm. The dynamic algorithm may be a machine learning algorithm, such as, e.g., a machine learning algorithm using a neural network. In embodiments wherein a dynamic algorithm is used, any of the components the mobility assessment is comprised of such as, e.g., any of the components described above may be generated or obtained using the dynamic algorithm. For example, in embodiments where the mobility assessment includes the detection of an anomaly as described above, the detection may be generated or obtained using a dynamic algorithm.

As discussed above, a mobility assessment can be generated for a subject from one or more MSK movement biomarkers. In some instances, the mobility assessment is generated in real time. By real time is meant the mobility assessment is generated during or immediately following generation of the visual recording. In some instances, the mobility assessment is generated in two hours or less. In some cases, the mobility assessment is generated in one hour or less, such as thirty minutes or less, or twenty minutes or less, or ten minutes or less, or five minutes or less, or one minute or less following generation of the visual recording.

In some instances, the mobility assessment is associated with an identifier of the subject. The mobility assessment and/or associated identifier may be saved to a database such as, e.g., a database including a data warehouse. In some instances, the data warehouse is used to determine a relationship between health or mobility outcomes, the diagnosis of a disease or condition, the fitness of a subject for performing a task, and one or more MSK movement biomarkers or mobility assessment components as discussed above. The relationship may be determined, at least in part, using a dynamic algorithm. The dynamic algorithm may be a machine learning algorithm, such as, e.g., a machine learning algorithm using a neural network. In some instances, the determined relationship may be used to generate a subsequent mobility assessment.

In some embodiments, the method further includes suggesting preventative measures based on the mobility assessment, such as, e.g., recommended equipment (e.g., braces) to avoid potential declines in mobility. In some embodiments, the method further includes providing a therapy recommendation to the subject based on the mobility assessment. While the therapy recommendation may vary, in some instances the therapy recommendation includes recommendations regarding the specifics of administering some existing standard of care for the treatment of a disease or condition. In some instances, the method further includes administering the treatment to the subject.

Embodiments of the methods may further include transmitting the mobility assessment, e.g., to a health care practitioner, to the subject, to an agent of the subject, etc. In some instances, the mobility assessment is received by a computer or mobile device application, such as a smart phone or computer app. In some cases, the mobility assessment is received by mail, electronic mail, fax machine, etc. Aspects of the invention further include methods of obtaining a mobility assessment, e.g., by using a system of the invention as discussed in greater detail below; and receiving a mobility assessment from the system.

### Telehealth Applications

Embodiments of the invention may further include computer or mobile device applications or software programs such as, e.g., smart phone or computer telehealth apps for practicing embodiments of the above methods. In some embodiments, the application or program allows the subject to customize one or more components of the above methods. For example, the application or program may allow the subject to choose the level of detail included in the instructions or the manner in which the instructions are conveyed as discussed above.

In some cases, the application or program allows the subject to select the activity including an oscillatory motion they wish to perform. In some embodiments, the application or program may provide the subject with a questionnaire used to determine one or more activities best suited for their specific circumstances. In some cases, the application or program may provide the subject with a selectable and filterable drop-down menu including a comprehensive list of all the activities the application or program is configured to assess (e.g., as discussed above) and the conclusions or assessments that can be made for each activity.

In some cases, the application or program allows the subject to select various components of the mobility assessment they wish to be provided. For example, in embodiments where the subject is a recreational weightlifter, the subject may select an activity including an oscillatory motion that resembles or is identical to a weightlifting movement the subject has experienced difficulty with. The subject may additionally only select a general assessment of the specific movement (e.g., as described above) to be included in the mobility assessment.

In some embodiments, the application or program saves previously generated mobility assessments and/or MSK movement biomarkers so that the subject can track how one or more metrics/assessments has changed for them over time. In some cases, the application or program allows a medical professional to give feedback or advice based on one or more mobility assessments and/or MSK movement biomarkers generated for the subject. In some embodiments, the application or program is configured to automatically and/or seamlessly perform one or more components of any of the embodiments of the methods discussed above.

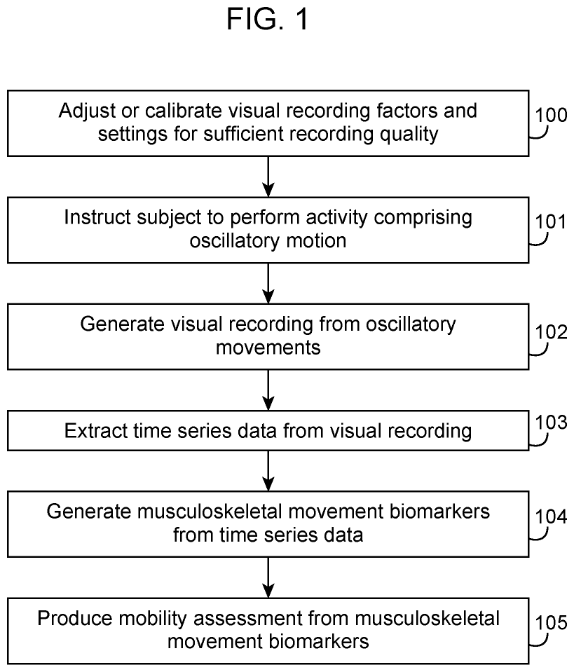

FIG. 1 provides a depiction of a method for generating a mobility assessment in accordance with an embodiment of the invention. At step 100, instructions guiding the subject through calibrating or adjusting one or more factors or settings affecting the quality of a visual recording are communicated to the subject through any number of various visual or audio means. The settings or factors adjusted or calibrated may include a lighting setting, one or more settings of the recording device, or the distance the subject is from the recording device. At step 101, the subject is instructed on how to perform the activity including the oscillatory motion, when to begin performing the activity, and when to cease or stop performing the activity. At step 102, a visual recording is generated of the subject performing the activity using a recording device. The generated visual recording may then be transmitted to a processor configured to perform additional steps of the methods. At step 103, time series data is extracted from the visual recording using a dynamic algorithm. The dynamic algorithm may include a deep learning algorithm such as a ResNet, InceptionNet, VGGNet, GoogLeNet, AlexNet, EfficientNet, or YOLONet neural network. Additional images may be labeled and used to train the dynamic algorithm in order to increase the accuracy of the time series data. At step 104, one or more MSK movement biomarkers are generated from the time series data. The one or more MSK movement biomarkers may be associated with an identifier of the subject and may be saved via local storage and/or cloud storage to a database. At step 105, a mobility assessment is produced for the subject from the one or more generated musculoskeletal movement biomarkers. The mobility assessment may be associated with an identifier of the subject and/or one or more of the MSK movement biomarkers and may be saved via local storage and/or cloud storage to a database. The mobility assessment may additionally be transmitted to a health care practitioner, the subject, or an agent of the subject.

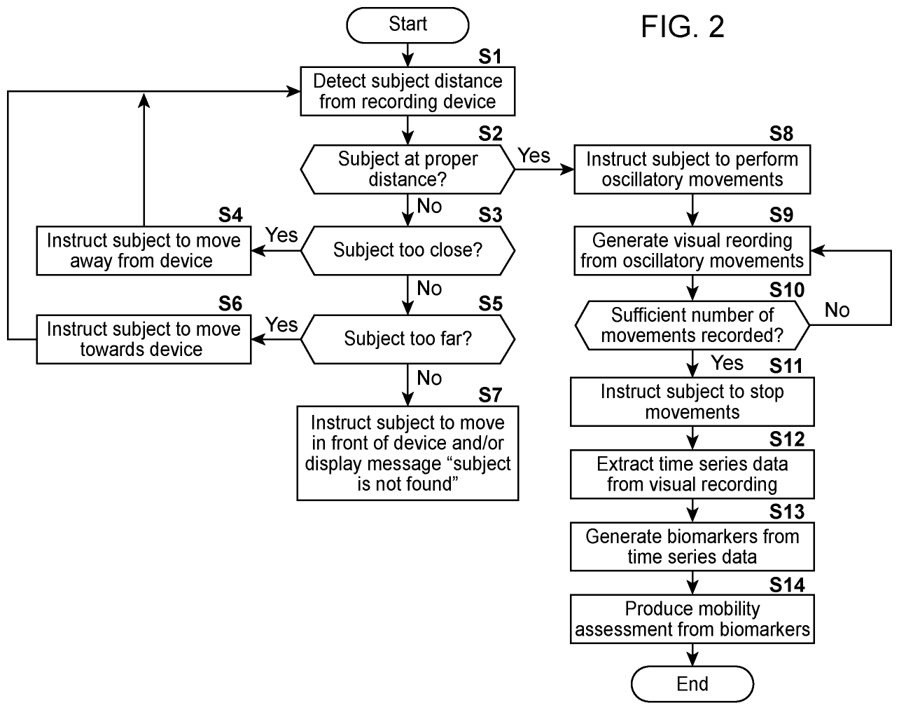

FIG. 2 provides a flow diagram depicting a method of instructing the subject in accordance with an embodiment of the invention. At step S1, a distance the subject is from the recording device is detected, e.g., based on automatically identified facial features or a LiDAR scanner. At step S2, it is determined if the distance is proper or, i.e., if the distance allows for the recording device to generate a visual recording of sufficient quality. If the distance is not proper, it is determined if the subject is too far or too close to the recording device at steps S3 and S4, respectively. If the subject is determined to be too far or too close to the recording device, the subject is prompted to take appropriate corrective actions at steps S4 and S6, respectively. If the distance is not proper, not too far, or not too close the subject is instructed to move in front of device and/or a visual display outputs the message “subject is not found” (S7). If the subject is determined to be a proper distance from the recording device, step S8 instructs the subject to perform the activity including an oscillatory motion or movement and a visual recording is generated the from oscillatory movements (S9) until a sufficient number of movements have been recorded (S10). Once it has been determined that a sufficient number of movements have been recorded, the subject is instructed to stop performing the movements at step S11. At step S12, time series data is extracted from the visual recording. MSK movement biomarkers are then generated from the time series data and a mobility assessment is produced from the biomarkers at steps S13 and S14, respectively.

### Systems

Aspects of the present disclosure further include systems, such as computer-controlled systems, for practicing embodiments of the above methods. Aspects of the systems include: a display configured to provide visual information instructing the subject to perform an activity including an oscillatory motion; a digital recording device configured to generate a visual recording of the subject performing the activity including an oscillatory motion; a processor configured to receive the visual recording generated by the camera; and memory operably coupled to the processor wherein the memory includes instructions stored thereon, which when executed by the processor, cause the processor to extract time series data from the visual recording using a dynamic algorithm, generate one or more musculoskeletal movement biomarkers from the time series data, and produce the mobility assessment for the subject from the one or more musculoskeletal movement biomarkers. The systems allow for a mobility assessment to be generated for the subject from a recording of the subject performing an activity including an oscillatory motion, as discussed above.

In some embodiments, the digital recording device may include any device capable of generating a sequence of visual images over time. In some embodiments, the recording device may include, but is not limited to, digital cameras or camcorders. In some embodiments, the recording device may be a smartphone camera or a computer camera (e.g., a webcam). For example, the recording device may be an iPhone camera, an Android camera, a personal computer (PC) camera such as, e.g., a tablet computer camera, a laptop camera (e.g., a MacBook or an XPS laptop camera), etc. In embodiments of the systems, the recording device is capable of generating a visual recording of sufficient quality. For example, the recording device may be capable of generating a visual recording having at least a minimum number of frames per second (FPS) or a minimum resolution. The minimum FPS or minimum resolution may vary, e.g., depending on the size of the subject or the oscillatory motion being performed by the subject. In some embodiments, the recording device is capable of producing a video having fifteen FPS or more, such as thirty FPS or more, or sixty FPS or more, or two hundred forty FPS or more, or five hundred FPS or more, or one thousand FPS or more, or fifteen thousand FPS or more. In some embodiments, the recording device is capable of producing a video having a resolution of 360p or more, such as 720p or more, or 1080p or more, or 2160p or more, or 4000p or more, or 4320p or more, or 8640p or more. In some embodiments, the recording device is capable of generating an audio recording (e.g., the recording device includes a microphone). In some cases, the system may further include a widget configured to stabilize the recording device such as, e.g., a tripod. In some embodiments, the recording device includes an audio recording component such as, e.g., a microphone.

In some embodiments, the system may further include a motion tracking marker or sensor configured to be worn or affixed to the subject. In some cases, the motion tracking marker or sensor is configured to be worn or affixed to a body part of the subject performing the oscillatory motion, as described above. The motion tracking marker or sensor may vary and includes, but is not limited to, a wearable device such as a smartwatch (e.g., Apple watches, Garmin watches, or Fitbit® watches). In some embodiments, the wearable device may include motion sensors (e.g., accelerometers, gyroscopes, and magnetometers), electrical sensors (e.g., electrocardiogram sensors), or light sensors (e.g., photoplethysmography (PPG) sensors). In some embodiments, the motion tracking marker or sensor is configured to produce a visual pattern or emit an audio frequency. For example, a smartwatch may be configured to display a striped pattern and/or emit a rapid series of audio chirps. The processor may include memory which, when executed by the processor, causes the processor to determine a distance the motion tracking marker or sensor is from the recording device or a distance the motion tracking marker or sensor has traveled between two sequentially generated visual images using, e.g., the resolution of the recording device and the number of pixels between components of the visual pattern. The processor may include memory which, when executed by the processor, causes the processor to determine a velocity the motion tracking marker or sensor is traveling away from or towards the recording device using, e.g., a change in the audio frequency (Doppler effect) or a change in the amount of time between audio chirps.

In some embodiments, the system may further include a device or machine configured to guide the movement of the one or more body parts of the subject in performing the oscillatory motion. In some cases, the device or machine may be configured to apply resistance. In some embodiments, the device or machine might include a sensor configured to generate information regarding the movement of the one or more body parts performing the oscillatory motion. In some embodiments, the device or machine may include a string or rope configured to be vertically pulled. In some embodiments, the device or machine might include a motion tracking marker that is configured to assist in tracking the position of one or more components of the device or machine. In other embodiments, the device or machine does not include a sensor or a motion tracking marker.

In embodiments of the systems where additional sensors or recorders are included in addition to the visual recording device, the processor is configured to receive data signals from the additional sensors or recorders. The display may be an electronic display device such as, e.g., a liquid crystal display (LCD), an organic light-emitting diode (OLED) display or an active-matrix organic light-emitting diode (AMOLED) display. In some embodiments, the display device may include a projector. In some cases, the display includes a loudspeaker configured to provide audio instructions to the subject.

In some embodiments, the memory includes instructions stored thereon, which when executed by the processor, further cause the processor to extract time series data using a dynamic algorithm. In some embodiments, the dynamic algorithm is a machine learning algorithm, such as a machine learning algorithm that uses an artificial neural network. For example, the artificial neural network may be a recurrent neural network (RNN), convolutional neural network (CNN), or region-convolutional neural network (R-CNN). The dynamic algorithm may include any algorithm capable of identifying one or more body parts of the subject. In some embodiments, the algorithm includes a deep learning algorithm such as, e.g., a ResNet, InceptionNet, VGGNet, GoogLeNet, AlexNet, EfficientNet, or YOLONet neural network. In some embodiments, the memory includes instructions stored thereon, which when executed by the processor, further cause the processor to train the dynamic algorithm using any data set that includes visual images labeled with one or more relevant body parts.

In some embodiments, the processor includes instructions stored thereon, which when executed by the processor, further cause the processor to generate one or more MSK movement biomarkers from the time series data according to any of the methods as discussed above. In some embodiments, the instructions, when executed by the processor, may cause the processor to produce a mobility assessment for the subject from the one or more generated MSK movement biomarkers according to any of the methods as discussed above.

In some instances the systems further include one or more computers for complete automation or partial automation of the methods described herein. In some embodiments, systems include a computer having a computer readable storage medium with a computer program stored thereon.

In embodiments, the system includes an input module, a processing module and an output module. The subject systems may include both hardware and software components, where the hardware components may take the form of one or more platforms, e.g., in the form of servers, such that the functional elements, i.e., those elements of the system that carry out specific tasks (such as managing input and output of information, processing information, etc.) of the system may be carried out by the execution of software applications on and across the one or more computer platforms represented of the system.

Systems may include a display and operator input device. Operator input devices may, for example, be a touchscreen, a keyboard, a mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C, C++, Python, other high-level or low-level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques. The processor may be any suitable analog or digital system. In some embodiments, the processor includes analog electronics which provide feedback control, such as for example positive or negative feedback control. In some embodiments, the feedback control is of, e.g., oscillatory movement performance.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described including a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid-state storage devices (including magnetic or optical disks or tape or RAM, or any other suitable device, either fixed or portable). The processor may include a general-purpose digital microprocessor suitably programmed from a computer readable medium carrying necessary program code. Programming can be provided remotely to processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (i.e., smartphone).

In some embodiments, systems according to the present disclosure may be configured to include a communication interface. In some embodiments, the communication interface includes a receiver and/or transmitter for communicating with a network and/or another device. The communication interface can be configured for wired or wireless communication, including, but not limited to, radio frequency (RF) communication (e.g., Radio-Frequency Identification (RFID), Zigbee communication protocols, Z-Wave communication protocols, ANT communication protocols, WiFi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, an RS-232 port, or any other suitable electrical connection port to allow data communication between the subject systems and other external devices such as a computer terminal (for example, at a physician's office or in hospital environment) that is configured for similar complementary data communication.

In one embodiment, the communication interface is configured for infrared communication, Bluetooth® communication, or any other suitable wireless communication protocol to enable the subject systems to communicate with other devices such as computer terminals and/or networks, communication enabled mobile telephones, personal digital assistants, or any other communication devices which the user may use in conjunction.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or WiFi connection to the internet at a WiFi hotspot.

In one embodiment, the subject systems are configured to wirelessly communicate with a server device via the communication interface, e.g., using a common standard such as 802.11 or Bluetooth® RF protocol, or an IrDA infrared protocol. The server device may be another portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touch-screen.

In some embodiments, the communication interface is configured to automatically or semi-automatically communicate data stored in the subject systems, e.g., in an optional data storage unit, with a network or server device using one or more of the communication protocols and/or mechanisms described above.

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include CSV, SQL, HTML or XML documents, email or other files, or data in other forms. The data may include Internet URL addresses so that a user may retrieve additional CSV, SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a workstation, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows, iOS, macOS, watchOS, Android, Oracle Solaris, Linux, IBM i, Unix, and others.

Aspects of the present disclosure further include non-transitory computer readable storage mediums having instructions for practicing the subject methods. Computer readable storage mediums may be employed on one or more computers for complete automation or partial automation of a system for practicing methods described herein. In certain embodiments, instructions in accordance with the method described herein can be coded onto a computer-readable medium in the form of “programming”, where the term “computer readable medium” as used herein refers to any non-transitory storage medium that participates in providing instructions and data to a computer for execution and processing. Examples of suitable non-transitory storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. A file containing information can be “stored” on computer readable medium, where “storing” means recording information such that it is accessible and retrievable at a later date by a computer. The computer-implemented method described herein can be executed using programming that can be written in one or more of any number of computer programming languages. Such languages include, for example, Python, Java, Java Script, C, C#, C++, Go, R, Swift, PUP, as well as many others.

The non-transitory computer readable storage medium may be employed on one or more computer systems having a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as those mentioned above, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

### Utility

The methods and systems of the invention, e.g., as described above, find use in a variety of applications where it is desirable to make a qualitative or quantitative determination regarding one or more mobility-related matters pertaining to a subject. In some embodiments, the methods and systems described herein find use when it is desirable to assess or diagnose MSK pathology. Embodiments of the present disclosure find use in applications wherein it is desired to acquire additional health and mobility information through non-invasive and remote diagnostic procedures in order to, e.g., facilitate the diagnosis of various diseases and conditions and, correspondingly, provide for improvements in patient outcomes. In some embodiments, the subject methods and systems may facilitate a determination regarding the recovery of a subject after an injury or surgery or the effectiveness of a method of treatment (e.g., physical therapy) through the generation of useful data by low or minimally trained technicians or without a technician. In some embodiments, the subject methods and systems may facilitate diagnosis for one or more conditions, insight on one or more health risks, or recommendations for one or more therapies or treatments.

### Examples

The following examples are put forth so as to provide those of ordinary skill in the art with a complete disclosure and description of how to use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Materials and Methods

### Animal Training Protocol and Behavioral Box Apparatus

All animal procedures were approved by the SFVA IACUC committee. 12 adult male wild-type mice (C57/L6J, Jackson Laboratory inc.) were trained on a string-pulling task in an acrylic box as described by Blackwell et. al. [11]. After 2 weeks of training, the mice were split into two surgical groups. Mice were placed in a plexiglass box (dimensions of 5×6×9 inches) for training and video recording of string pulling behavior. 3D printed string holders were used to standardize string placement in each box.

### Iatrogenic Rotator Cuff Injury

One group (n=6) underwent a right supraspinatus (SS) and infraspinatus (IS) tendon transection and denervation (TTDN) while another group (n=6) underwent right SS and IS TTDN with immediate repair of the torn tendons as described by Wang et al. [28]. String pulling behavior was recorded for all mice at their preoperative baseline and at postoperative weeks 1, 2, 3 and 4. Prior to each recording on the postoperative weeks 1-4, mice were given a brief string pulling training session as a reminder of the task requirements.

### Video Recordings:

A 1920×1080 HD video camera recording at 59.96 frames-per-second was used to acquire string pulling videos. The location of the camera relative to the plexiglass box was fixed across sessions using an alignment jig. Video recordings (of approximately 15-30 seconds in length for each pulling trial) were then trimmed with ffnpeg v 4.4.2 to contain only the string pulling behavior.

### Kinematic Segmentation:

The X/Y coordinates of each hand were acquired with DeepLabCut (DLC) v 2.2.0 using a ResNet50 deep convolutional neural network model. Two DLC models were built across the experiment: 1) a pilot cohort of 3 mice and 2) a full model that included data from all 13 mice pooled together (including mice in the pilot experiment). The locations of the right and left hands were labeled for 320 and 1080 video frames across the pilot and full models, respectively. After initial training, an extra 160 and 1440 frames were extracted as outliers (based on a criteria of ≥10 pixel jumps in Euclidean distance between consecutively labeled points across video frames) for the pilot and full models, respectively. Each iteration of the ResNet50 model was trained for 200,000 iterations and the resultant mean Euclidean error in label location (determined on a held-out set of test images consisting of 5% of the frames in each training set) was calculated using the built-in DLC function evaluate network. A total of 27 videos were used for neural network training; all training was performed using an NVIDIA 2080 Ti GPU with default image augmentation enabled.

### String Pulling Trajectory Trace Post-Processing

Once the X/Y coordinates of each hand were segmented using DLC, the resulting traces were highpass filtered with a first-order 0.75 Hz Butterworth filter (to remove trajectory drift from minor postural changes across the pulling cycle) and then lowpass filtered with a third-order 9 Hz Butterworth filter (to remove occasional jitter in trajectory segmentation). All subsequent analyses are performed using filtered data. After filtration, the peaks/troughs in the Y-axis (vertical direction) hand trajectory trace were labeled using SciPy's find peaks function.

### Calculation of Amplitude and Time for Reach/Pull Epochs

Reach epochs were defined as the time between each trough and its successive peak in the Y-axis kinematics trajectory; pull epochs were defined as the time between each peak and its successive trough in the Y-axis kinematics trajectory. For every reach/pull, the amplitude (in pixels) was measured between each successive trough-to-peak and peak-to-trough epoch corresponding to every reach and pull, respectively. Reach/pull time was measured in number of videos frames, divided by the video framerate, between each successive trough-to-peak and peak-to-trough epoch corresponding to every reach and pull, respectively.

### Calculation of the Full Width at Half Maximum

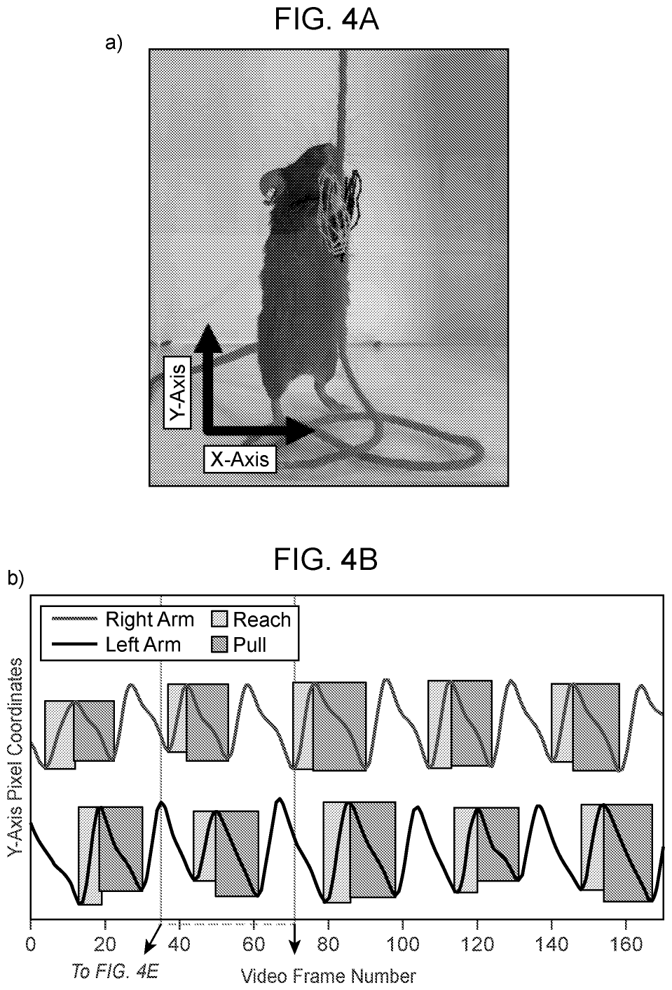

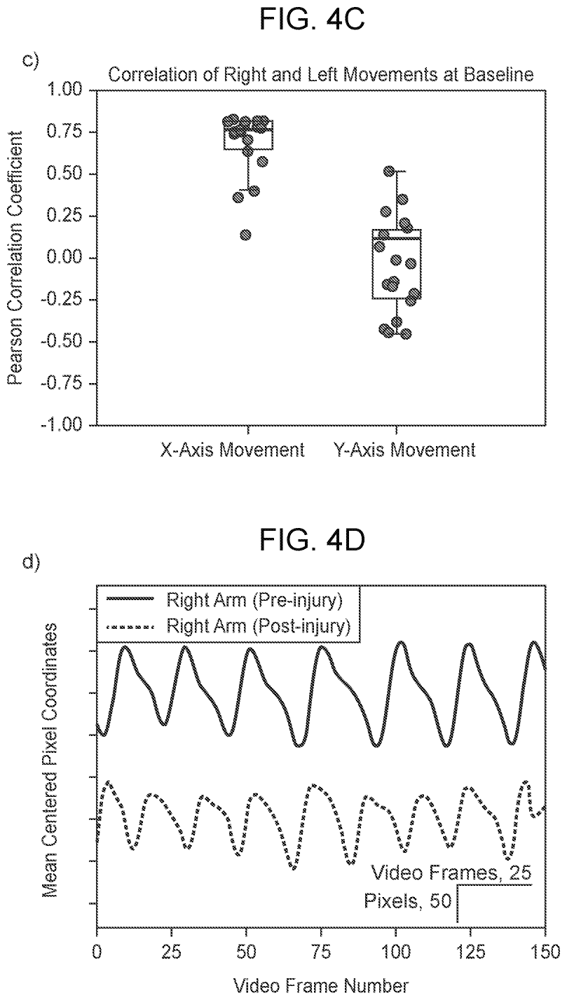

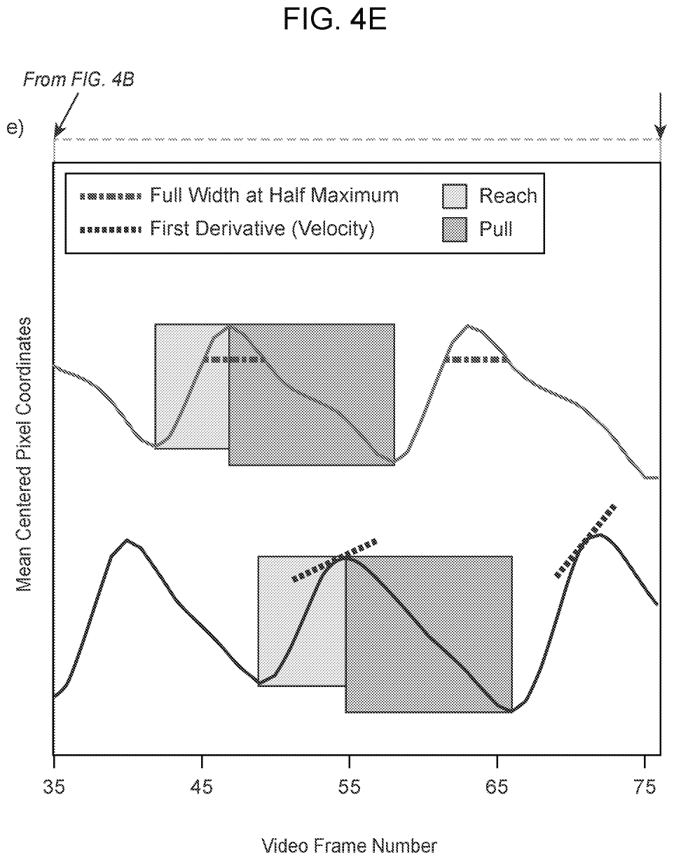

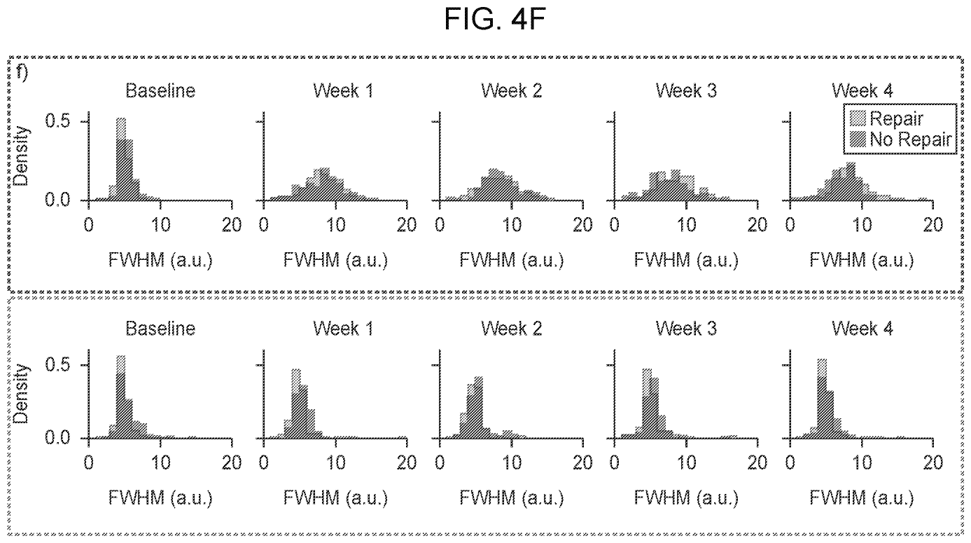

In order to quantify the shape of the string pulling waveform across the experimental timeline, the full width at half maximum (FWHM) was calculated as a hybrid measure of movement fluency during the period of behavior covering both reaching and pulling. To calculate the FWHM, the Y-axis kinematics trace for the right and left hands was mean-centered and the periods of the pulling trajectory between the signals' negative-to-positive and positive-to-negative zero-crossing was extracted for analysis. Each epoch was interpolated using a 100 point 2^{nd}-degree univariate spline, and the FWHM was calculated as the width (in fractional video frame number) of each peak at 1% its vertical amplitude. (See FIG. 4E, FWHM values (black lines) are overlaid on a representative string pulling waveform).

### Calculation of Velocity and Acceleration

To calculate velocity and acceleration of the right and left hands across the transition from reaching to pulling, the Y-axis kinematics trace for the right and left hands was processed as described in the section on calculating the full width at half maximum of the signal. After extracting the interpolated signal, the first and second derivates were taken as measures of velocity and acceleration, respectively. (See FIG. 4E, velocity values (green lines) are overlaid on a representative string pulling waveform).

### Correlation in Hand Movement

To measure the consistency of string pulling behavior across the right and left hands, the Pearson's correlation coefficients were calculated by correlating X_{right} with X_{left} and Y_{right} with Y_{left} kinematic traces (all correlations were run after data was high pass and low pass filtered). All correlations were run within animal and within day.

### Quantifying Kinematic Synergies Using Principal Component Analysis

The shoulder is a complex joint that allows for multiplanar motion across arm flexion, extension, abduction, adduction, internal rotation, and external rotation. Moreover, shoulder motion is intimately tied to scapular and thoracic mobility as both contribute to stabilization of the upper extremity across its full range of motion; in total, about 20 skeletal muscles contribute to shoulder motion [6]. Prior research on the human hand, has shown significant biomechanical and temporal linking across joints during various hand movements thus suggesting that the biomechanical and neural representations of the hand are significantly lower dimensional than the degrees of freedom conferred by individual muscles and joints would imply [20][21].

The kinematic synergies were studied by performing singular value decomposition (SVD) independently on each string pulling epoch from individual mice across weeks. In brief, the filtered kinematics trace containing data for the right hand X-axis movement, right hand y-axis movement, left hand x-axis movement, and left hand y-axis movement were mean-centered and then concatenated into a matrix T∈R^{4×t} with t representing the number of video frames in each video recorded from a given mouse on a given week.

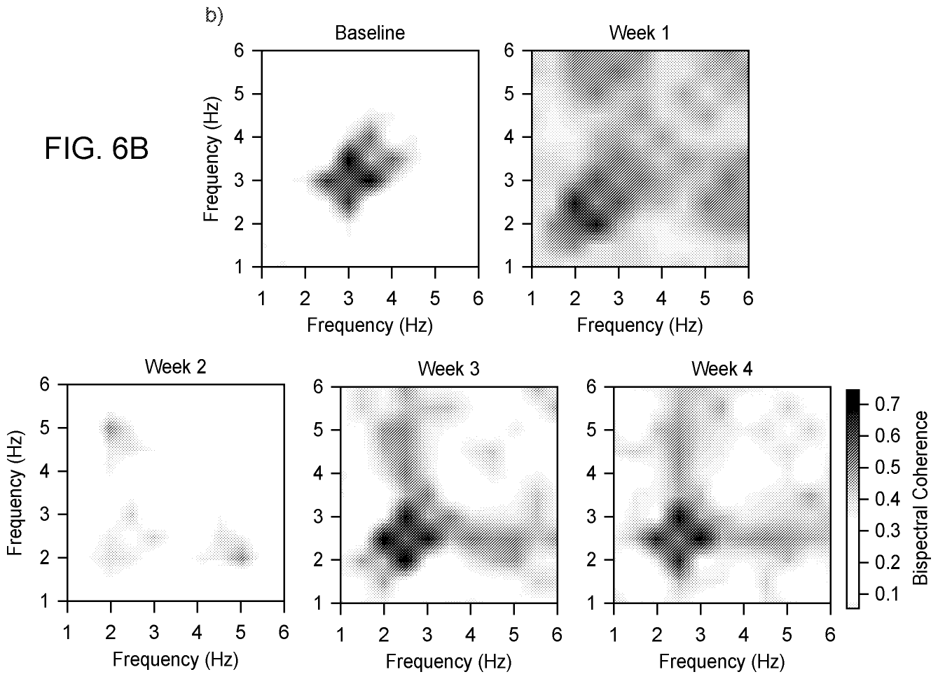

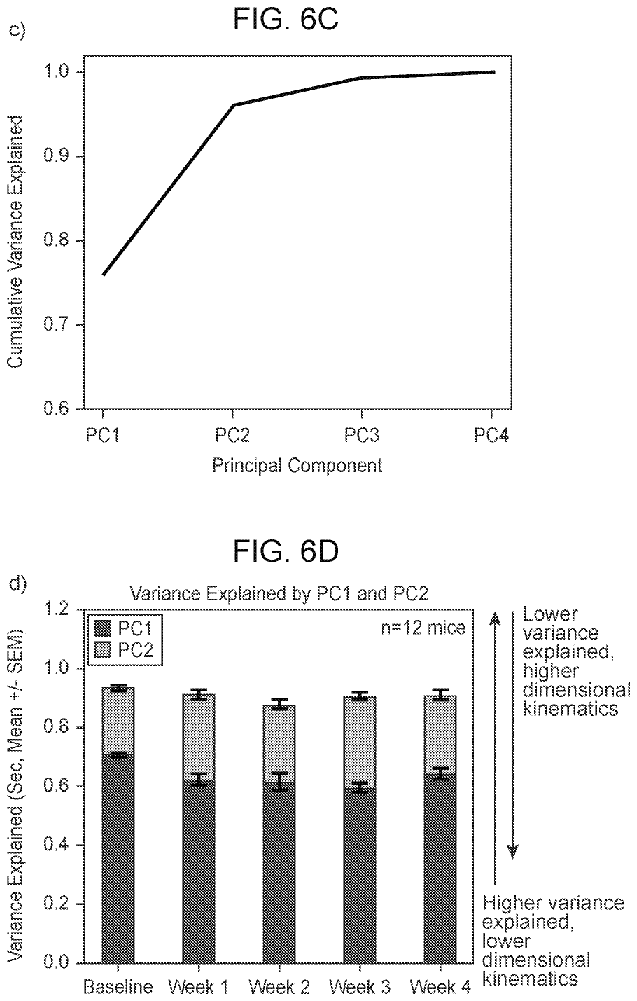

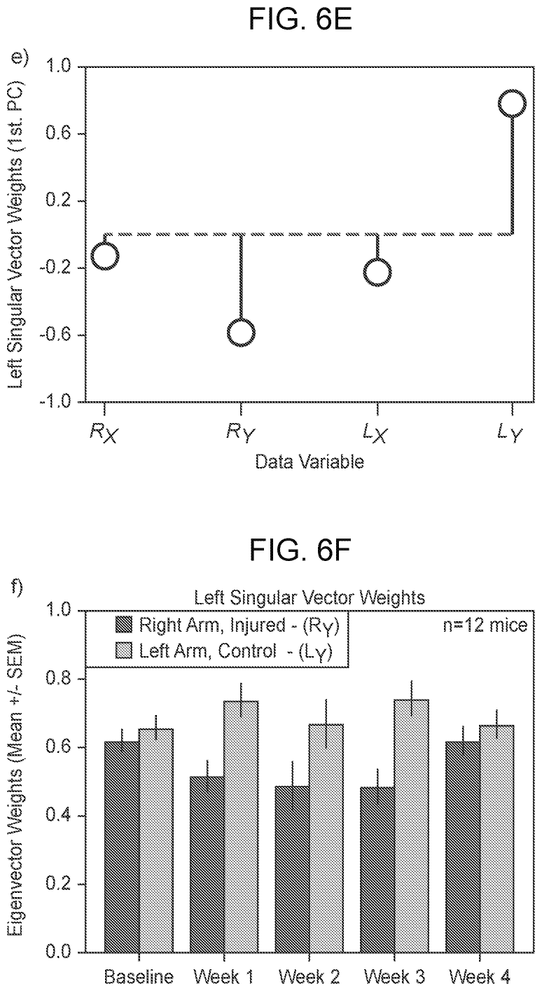

The matrix T was decomposed using SVD into the matrices U∈R^{4×4}, S∈R^{4×4}, and V^{T}∈R^{4×t}. The columns (i.e., principal components) of U capture the covariation patterns between the four tracked kinematic variables, with the individual weights of each column capturing both the magnitude and direction with which each kinematic variable contributes to that particular component. The absolute value of the weights in the first principal component were used to capture the magnitude of these kinematic variables for right and left hand Y-axis movement in FIG. 6E. The percent variance explained by each principal component is calculated by squaring the singular values in matrix S and then dividing each squared singular value by the sum of all squared singular values (reported in FIG. 6D for the first two PCs). The variance explained by each PC has previously been shown to correlate with the dimensionality of the kinematic synergies, with lower variances explained by each individual PC corresponding to a higher dimensional kinematic synergy as more PCs are required to reach the same cumulative proportion of explained variance. Lastly, each row of the matrix V^{T} captures the relative temporal contribution of each principal component across each video recording.

### Bispectral Coherence (Bicoherence) Analysis

In order to understand the temporal relationship between activation of PC1 and PC2, bicoherence was used to measure the cross-frequency coupling between the right singular vectors corresponding to PCs 1 and 2. In brief the scipy ‘spectrogram’ function was used to take the time-frequency decomposition of right singular vectors of PCs 1 and 2 using an FFT window length of 2 seconds with 1 second of overlap. The bicoherence analysis was then performed as described elsewhere²³. Because individual videos contained behavioral epochs of differing lengths, the resulting bicoherence values were linearly interpolated between 0-15 Hz in 0.1 Hz for each subject.

### Human Data Recordings

All patients gave their informed consent to participate in the study protocol as approved by the UCSF Committee on Human Research and all procedures were approved by the UCSF Institutional Review Board (IRB). Control and rotator cuff injury patients were recruited through convenience sampling at the UCSF Orthopedic Institute. Study participants were given minimal instruction on how to perform the string pulling task by the lead study author (D.D.) and then allowed to string pull at their own preferred rhythm and kinematic preference. Video was recorded using a tripod-mounted smartphone camera (iPhone 13 Pro Max) set two meters away from each subject. Video was recorded in HD resolution (1920×1080) at 59.94 frames per second. The resulting videos where subsequently processed in DeepLabCut using XYZ training frames and two successive refinement steps (outlier frames were defined as those frames with a Euclidean distance between two successively labeled points of ≥20 pixels). Both the hands and the elbows were labeled for the human recordings as the elbows were readily visible human subjects vs. rodents where the elbows are hidden by a layer of fur. For subjects in the injury cohort, the arm with the rotator cuff tear was labeled as in the “injured” extremity while the contralateral arm was labeled as the “uninjured extremity.”

### Human Data Pre-Processing

In order to ensure best performance for the detection of peaks/troughs in the analysis of string pulling amplitude & time, the Y-axis kinematic trajectory for the hands was highpass-filtered at 0.1 Hz with a 1^{st} order Butterworth filter followed by a 7^{th} order Butterworth 7 Hz lowpass filter. The peaks/troughs of this signal were than extracted and analyzed analogously as for rodents, described in the section “Calculation of Amplitude and Time for Reach/Pull Epochs” above. For all other analyses (including calculation of the full width at half maximum, velocity, acceleration, and PCA decomposition) the X/Y kinematic trajectories were not highpass filtered for the hands and elbows. Instead only performing lowpass filtering was used as the human data was less noisy when compared to the rodent data. Methods for calculating the full width at half maximum, velocity, acceleration, and PCA decomposition of the signals were performed analogously to the methods used for analyzing rodent data (see sections “Calculation of the Full Width at Half Maximum”, “Calculation of the Velocity and Acceleration”, and “Quantifying Kinematic Synergies Using Principal Component Analysis” above).

### Movement Dynamic Range Ratio

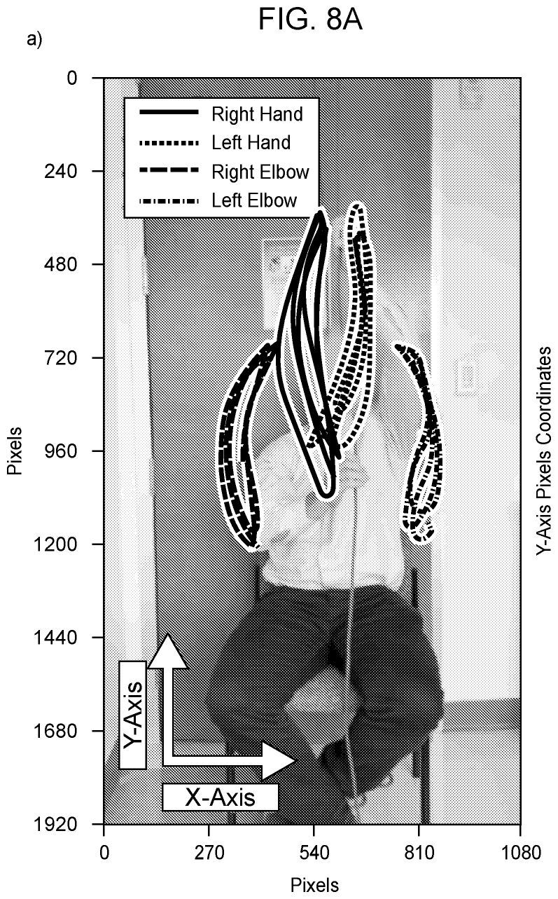

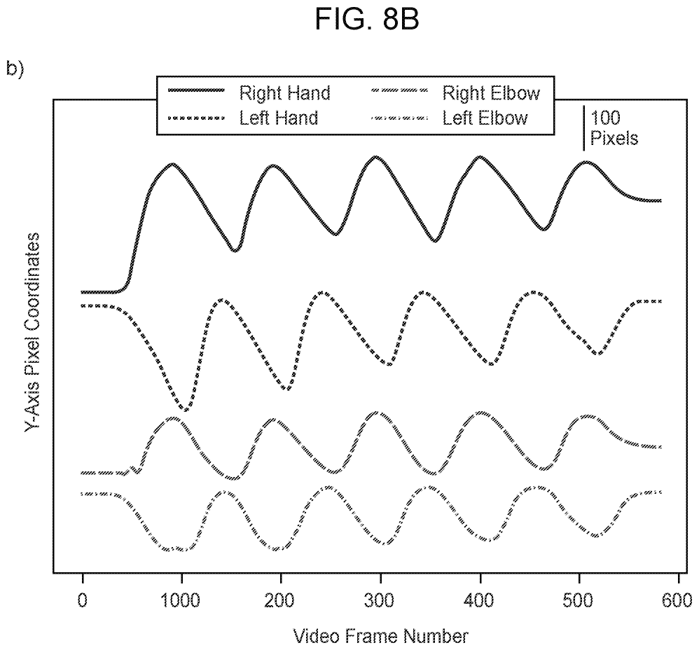

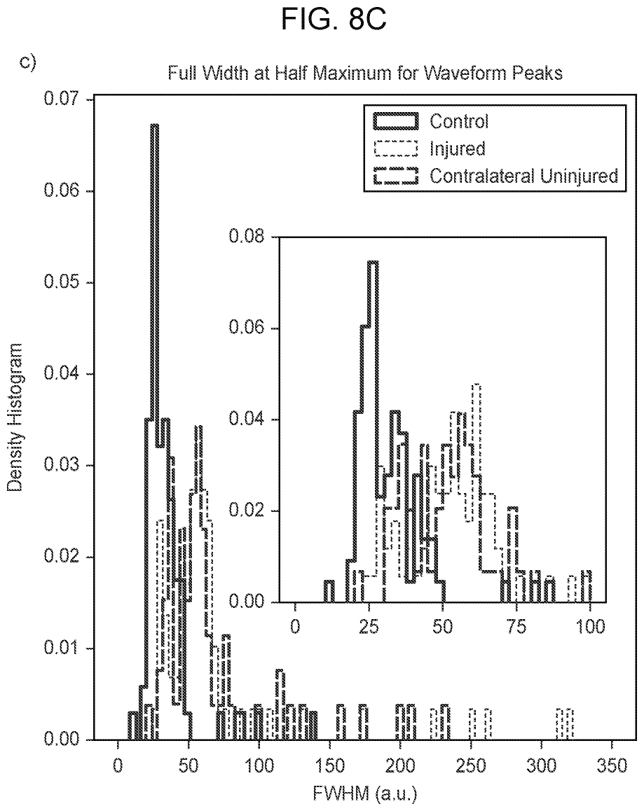

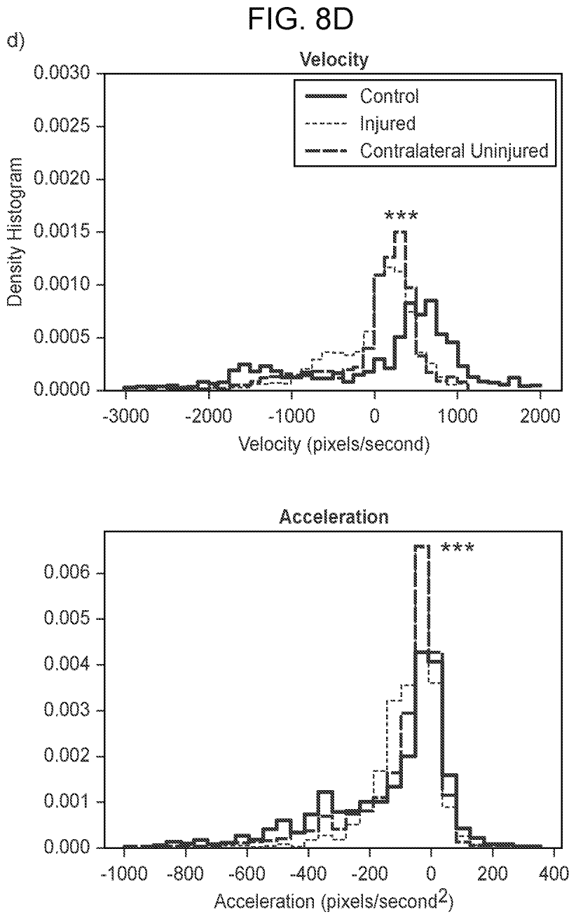

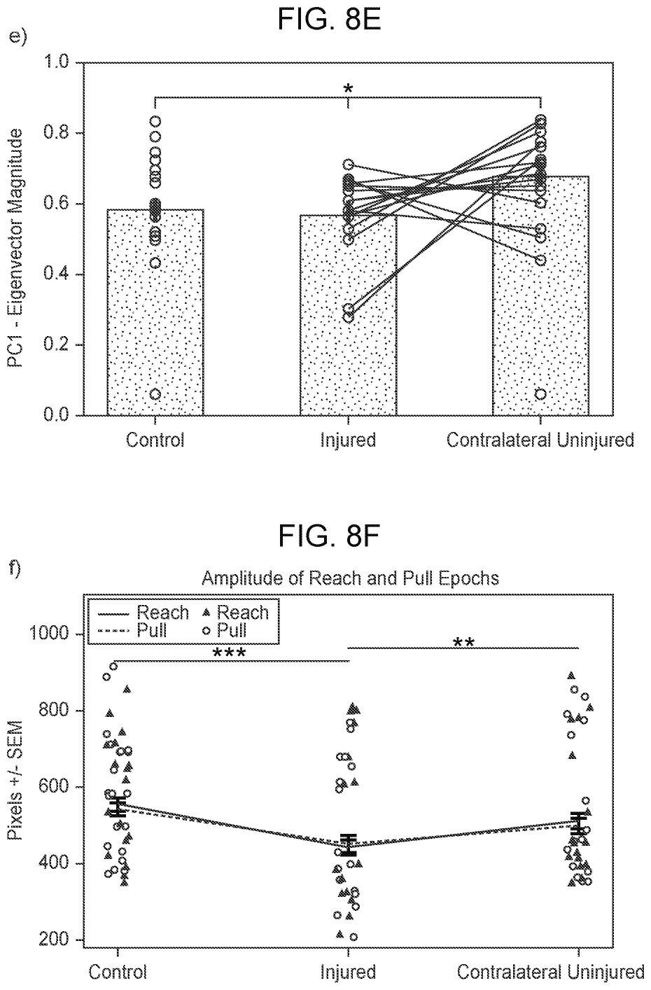

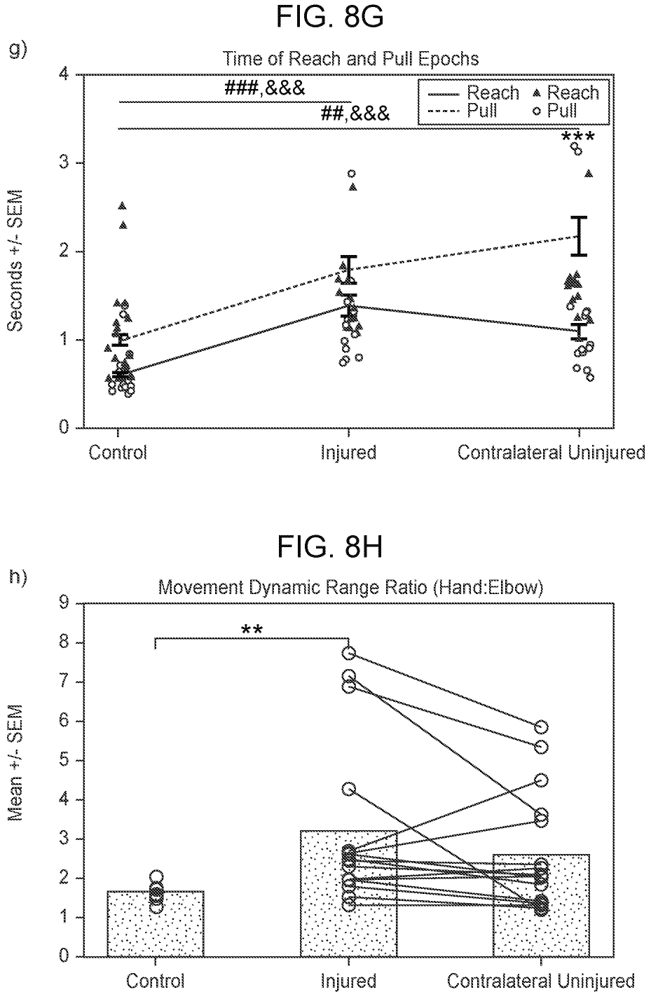

Because it is possible to track the position of both the hands and the elbows in human subjects, a Movement Dynamic Range Ratio was calculated that quantified the relative contribution of hand vs elbow movements in the string pulling behavior. The Movement Dynamic Range Ratio was calculated by first taking the standard deviation of the lowpass filtered Y-axis kinematic trajectories for each subject's hands and elbows. The within-subject ipsilateral ratio between the two standard deviation values (e.g. Std. Dev. Right Hand/Std. Dev. Right Elbow) was then taken across the right/left hand and elbow. These values were than reported as mean+/−SEM in FIG. 8h. Intuitively, if subjects predominately moved the arm from the shoulder as the main pivot point, the elbows and the hands would exhibit roughly the same vertical displacement in space (i.e. the Movement Dynamic Range Ratio would be close to 1; see FIG. 8h, control shoulders). On the other hand, if subjects immobilize the shoulder and instead move the arm through rotation of the humerus around its longitudinal axis, it is expected that the vertical displacement of the hands will exceed the vertical displacement of the elbows (i.e. the Movement Dynamic Range Ratio would be greater than 1; see FIG. 8h, injured shoulders).

### Statistics

Linear mixed-effects (LME) models were used to test the significance of differences in means across weeks and rotator cuff repair status. Using these models accounts for the fact that kinematics from the same animal are more correlated than those from different animals; thus, it is more stringent than computing statistical significance over all subjects [33]. LME models were fit using statsmodels v 0.13.2 and included a random intercept for each mouse. Levene's test was used to test for differences in variance across weeks. All analyses were performed using Python v 3.10.5.

### Results

### Behavioral Apparatus and Data Collection Methodology.

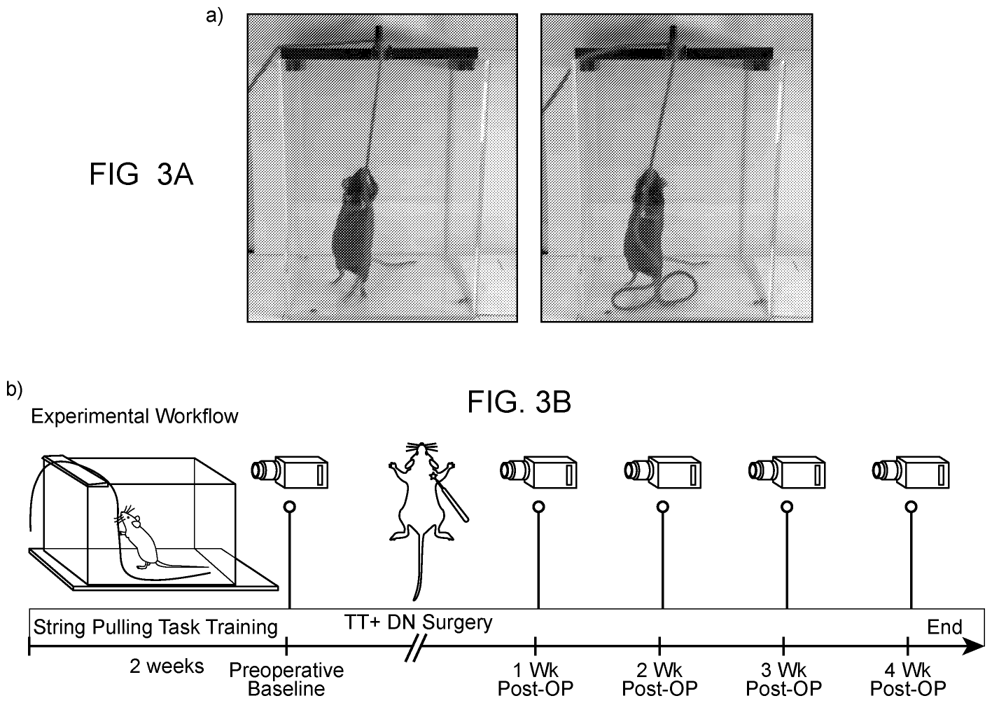

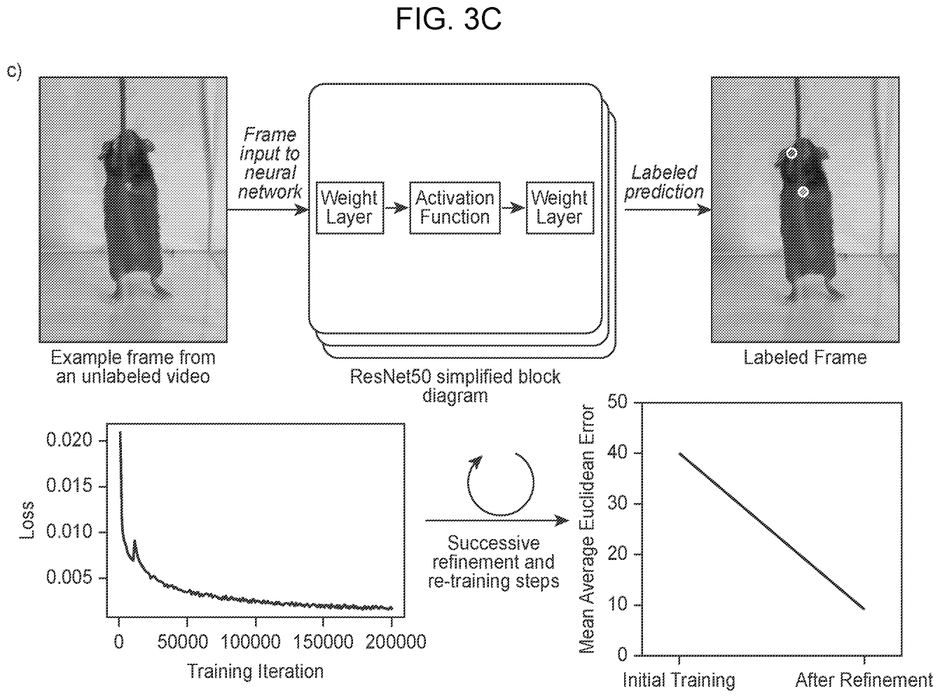

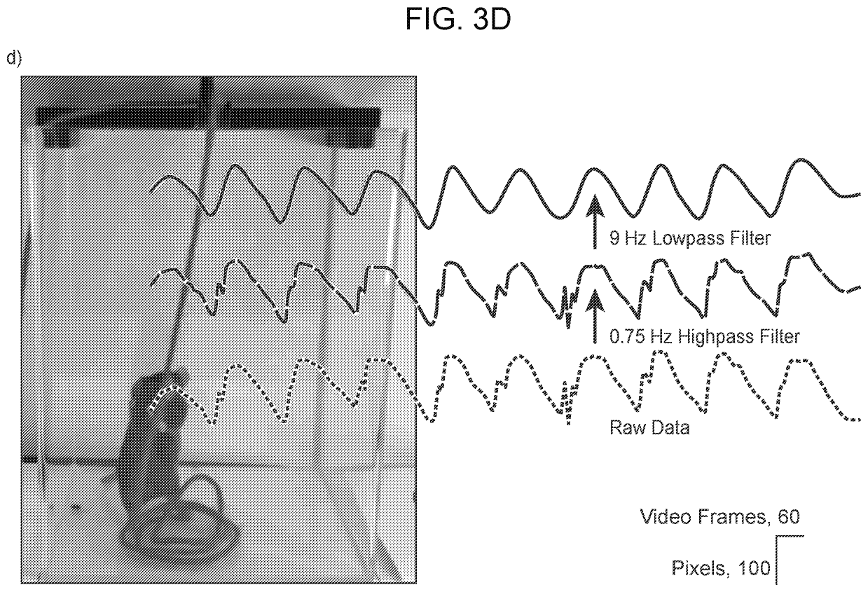

Mice were acclimated to a plexiglass behavior box for two days prior to the start of string pulling training. A 3D printed string holder placed over the behavior box standardized the position of the string across animals and behavioral bouts (FIG. 3a). The position of the video camera relative to the behavior box was standardized across recordings by using an alignment jig. Following acclimation, mice (n=12) received two weeks of string pulling training conducted three times per week (FIG. 3b). This was followed by a preoperative baseline behavioral recording where each mouse was recorded pulling a 0.75 m long string for a total of two trials (˜20-30 sec of data per animal; see Supplementary Movie 1 for representative baseline string pulling behavior). Animals then underwent surgery with combined supraspinatus (SS)/infraspinatus (IS) tendon transection and denervation of the right shoulder; half of the animals (n=6 mice) received immediate repair of the SS/IS tendons [9], [10]. After animals recovered for one week, string pulling behavior was recorded for an additional four weeks. After completion of data collection, DeepLabCut, a package for training deep convolutional neural networks, was used for automated image segmentation [19], to extract locations and labels of the right and left hands (FIG. 3ci). In brief, 50 video frames were extracted from each recorded video and labeled by manual curation. These videos were then used for supervised transfer learning of a ResNet50 deep convolutional neural network (CNN) that was pre-trained on ImageNet (FIG. 1c, middle). Feed forward inference was then performed on all video frames in the dataset to automatically label the right and left hands (FIG. 3ci, left). After an initial round of CNN training and inference, frames where the labels for at least one of the hands jumped by a Euclidean distance of 20 or more pixels were extracted. These frames were relabeled and the CNN retrained; this refinement step reduced the mean Euclidean error in label prediction from 40.16 to 9.21 pixels on a randomly selected 5% set of held-out test images (FIG. 3cii, right). On rare occasions, a mouse's hands would be occluded by the string causing brief oscillations in the labeling (see FIG. 3d, “Raw Data” trace for a worst-case scenario example). Accordingly, the hand trajectories were filtered first with a 0.75 Hz 1st-order Butterworth high pass filter (to remove any contributions from low frequency postural changes) followed by 9 Hz 3rd-order Butterworth low pass filter (to remove any oscillations in hand labeling secondary to hand occlusion). Filter frequencies and orders were selected to minimize distortion of the kinematic trajectory.

### Post-Processing of String Pulling Trajectories.

FIG. 4a demonstrates the overlay of 10 cycles of string pulling behavior for an example mouse prior to injury (right arm in blue, left arm in red). It was found that the oscillatory nature of the behavior with movement trajectories occurred predominately in the vertical Y-axis. Temporally unrolling the Y-axis trace revealed that reach epochs evolved faster than pull epochs as the latter required mice to apply downward force as they advance the string (FIG. 4b). For each reach and pull epoch, the duration (in number of video frames divided by frame rate) and amplitude (measured in number of pixels) were calculated. At baseline, Pearson correlations of the filtered kinematics traces for the right vs left hand in the X and Y axes revealed a high correlation in side-to-side (X-axis) movements of the arms (Median, ρ=0.7660, Q1=0.6245, Q3=0.8239, IQR=0.1994) while movements in the Y-axis advance (Median, ρ=0.0496, Q1=−0.1381, Q3=0.4331, IQR=0.5712) were anticorrelated, which is expected given that the arms oscillate out of phase as mice alternate reach and pull epochs to advance the string. After iatrogenic injury to the SS/IS tendons, qualitative changes to the shape of the Y-axis string pulling waveform including decreased velocity of pulls as well as “rounding” of the waveform peak at each reach-to-pull transition were observed (FIG. 4d, same mouse as in FIG. 4a). In order to quantify the coordination across the reach-to-pull transition, the full-width at half maximum (FWHM) of each peak in the waveform was calculated (FIG. 4e, example FWHM calculation, black lines, shown in the top trace). The velocity and acceleration of the arms was also calculated by taking the first and second derivates of the Y-Axis kinematic trace data, respectively. (FIG. 4e depicts an example velocity calculation, green lines, shown in bottom trace).

### RC Injury Impairs Movement Coordination and Dynamic Range.

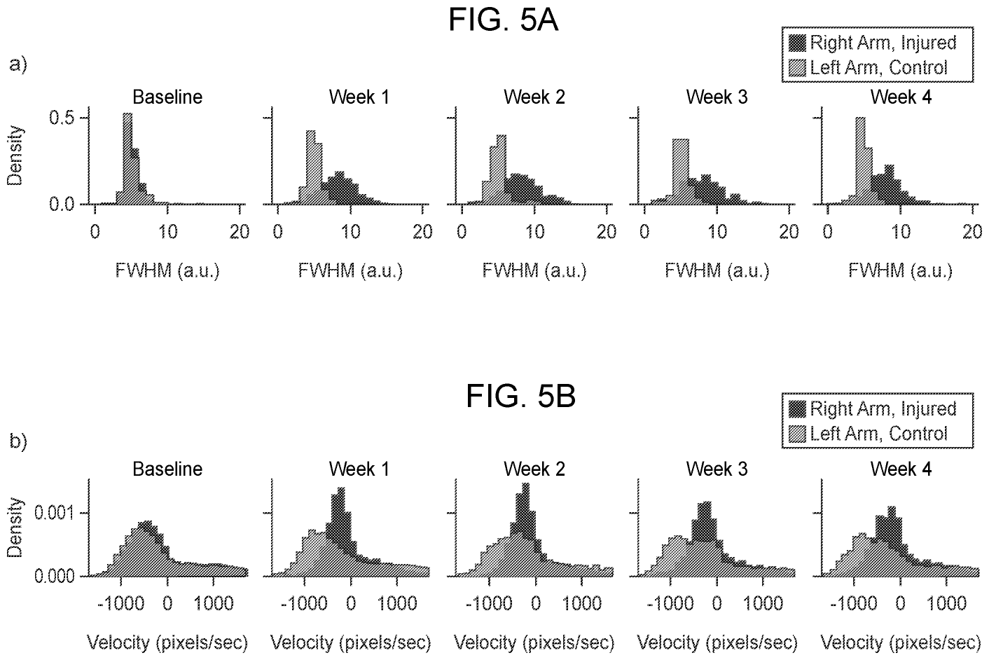

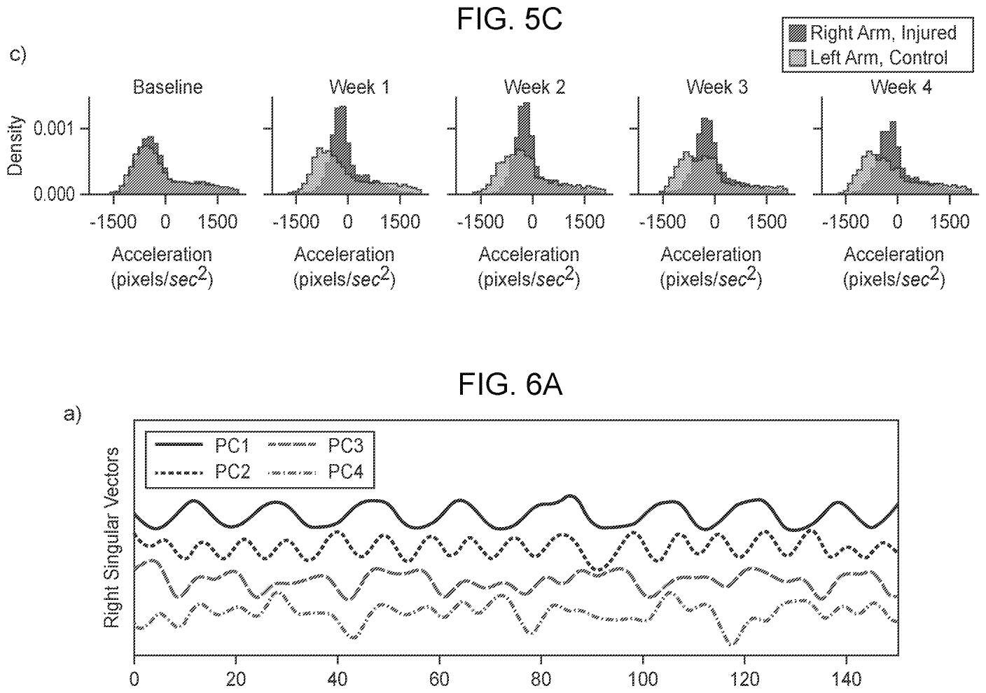

Initially, determining if immediate repair of the RC after iatrogenic injury would accelerate healing was of interest. Analyzing the FWHM measure (FIG. 4e) for the right hand (light blue background) and the left hand (light red background) revealed no differences in waveform shape for animals with and without immediate RC repair (two-sided Kolmogorov-Smirnoff test, right hand repair vs no repair: test statistic=0.0576, p-value=0.2001; two-sided Kolmogorov-Smirnoff test, left hand repair vs no repair: test statistic=0.2069, p-value=0.3692; n=6 mice with no repair and n=6 mice with immediate repair). The repair and no-repair groups were collapsed together for all further analysis. Examining the FWHM between the Baseline and Week 1 recordings revealed a striking post-injury rightward shift in the values suggesting that animals progress slower across the reach-to-pull transition (FIG. 5a). A two-way ANOVA revealed that there was a statistically significant interaction between the main effects of arm (injured vs control) and time (F(4, 2800)=99.97, p-value=1.2*10⁻⁷⁹). In contrast to changes in FWHM, the distribution of the velocity values for the injured arm exhibited a smaller relative change (FIG. 5b). Specifically, decreased density of negative velocity values, which is consistent with qualitative observations in FIG. 4d, suggested that the pull phase is especially affected by decreased slope magnitude. Despite this shift in density, a two-way ANOVA reveals no change in the central tendency of the mean for velocity (main effect of time, F(4, 53380)=0.504, p-value=0.733; main effect of arm (injured vs control), F(1, 53380)=0.195, p-value=0.659; interaction between time and arm, F(4, 53380)=0.832, p-value=0.505). Similar to velocity, a drop in the density of negative accelerate values (FIG. 5c) was noticed. This finding again suggests that, after undergoing RC injury, mice were unable to generate rapid arm motions especially in the downward direction. However, in contrast to velocity, where there was no change in central tendency of the mean, acceleration exhibited a significant change in mean value across the experiment with both a main effect of arm (injured vs control) as well as time (main effect of time, F(4, 50570)=3.878, p-value=3.878*10⁻³; main effect of arm (injured vs control), F(1, 50570)=13.437, p-value=2.469*10⁻⁴; interaction between time and arm, F(4, 50570)=26.067, p-value=1.277*10⁻²¹). It was also found that neither FWHM, velocity, or acceleration recovered across the four post-operative weeks, suggesting lasting deficits in end-effector output secondary to rotator cuff injury.

### Kinematic Synergies are Disrupted by Injury and Gradually Recover Across Time.

Recent research has suggested the brain encodes a low dimensional manifold to decrease the complexity of actuating hDOF joints [21], [22]. These low dimensional manifolds, otherwise known as synergies, manifest as synchronous EMG activity in skeletal muscle and finally through coordinated limb movement itself [22]. Here principal component analysis (PCA) was used to identify low-dimensional covariation patterns in high-dimensional data by decomposing the X-/Y-axis movement traces of the right and left arms into kinematic synergies that are expressed as low dimensional principal components. The right singular vectors generated as part of the PCA algorithm capture the time evolution of each principal component across a video recording. In a representative example at baseline, clear 2-3 Hz coupling was noticed between the right singular vectors of PC1 and PC2 (FIG. 6a)—in other words, for every one oscillatory cycle of PC1, PC2 exhibits two-to-three oscillatory cycles. Because the cumulative variance explained by both PC1 and PC2 was >95% at baseline, analyses was focused only on the first two PCs. Thus, to quantify the relationship between the right singular vectors of PCs 1 and 2, a bispectral coherence analysis was performed [23]. Bispectral coherence quantifies cross-frequency coupling between two time series; strong coupling was found between PCs 1 and 2 during the pre-injury baseline (FIG. 6b, mean bispectral coherence values shown across n=12 animals). Following injury, the cross-frequency coupling degraded between PC1 and PC2 across post-injury weeks 1 and 2. Bispectral coherence then remerged at post-injury weeks 3 and 4 suggesting restoration of temporal coupling between PCs 1 and 2.

In addition to tracking cross-PC coupling, the cumulative variance was also examined as explained by the principal components. In general, the higher the variance explained by a smaller number of PCs, the lower dimensional the kinematic synergy (and vice versa). FIG. 6c shows the cumulative variance explained by the four PCs for a representative mouse during the pre-injury baseline (same animal as shown in FIG. 6a). It was noticed that the first two PCs account for 96% of the variance explained. After RC injury, the variance explained by PC1 decreased from its peak at baseline (FIG. 6d) before increasing again at four week's recovery (Variance explained by PC1, Mean±SEM, n=12 mice: Baseline 0.707±0.007, Week 1 0.624±0.019, Week 2 0.616±0.029, Week 3 0.600±0.015, Week 4 0.645±0.021). Meanwhile, the variance explained by PC2 increased from its minimum value at baseline (variance explained by PC2, Mean±SEM, n=12 mice: Baseline 0.229±0.007, Week 1 0.289±0.017, Week 2 0.265±0.017, Week 3 0.310±0.011, Week 4 0.267±0.018). A one-way ANOVA revealed a statistically significant effect of week on variance explained by PC1 (F(4,75)=5.950, p-value=3.258*10⁻⁴) and a Tukey multiple comparison corrected post-hoc analysis showed no statistically significant difference between the variance explained at Baseline and Week 4 for PC1 (Baseline vs Week 4, p-value=0.093). For PC2, a one-way ANOVA also revealed a statistically significant effect of week on variance explained PC1 (F(4,75)=4.589, p-value=2.278*10⁻³). However, a Tukey multiple comparison corrected post-hoc analysis showed no statistically significant difference between the variance explained by PC2 for Weeks 2 and 4 when compared with Baseline (Baseline vs Week 2, p-value=0.546; Baseline vs Week 4, p-value=0.347).

While quantifying the percent variance explained by the first two PCs provides insight into the dimensionality of the kinematic synergy governing movements of the right and left arms, it does not provide insight into how the right and left hands are individually contributing to the synergy. By examining the eigenvectors of the PCA decomposition, insight can be gained into the magnitude and direction with which each of the four kinematic variables (Right arm X-axis (R_{X}), Right arm Y-axis (R_{Y}), Left arm X-axis (L_{X}), and Left arm Y-axis (L_{Y}) trajectories) contribute to each PC. FIG. 6e shows a stem plot of the eigenvector weights for the PC1 for a representative animal at baseline (same animal as shown in FIG. 6a,c). Here it was found that movements of the right and left arms in the Y-axis were similar in magnitude but opposite in sign—as expected given that the arms oscillate out of phase during the string pulling behavioral cycle (FIG. 4b,c). Since the sign of an eigenvector is relative, the absolute value of the eigenvector weights was taken for R_{Y} and L_{Y} to compare changes in weights across time. At Baseline, both R_{Y} and L_{Y} had similar magnitudes before diverging on Weeks 1-3. This divergence was followed by a convergence of weights on Week 4. A two-way ANOVA revealed a statistically significant effect of the interaction between week and arm on absolute eigenvector weights (Main effect of week, F(4,150)=0.918, p-value=0.455; Main effect of arm, F(1,150)=0.352, p-value=0.554; Interaction of week and arm, F(4,150)=2.652, p-value=0.045; n=12 mice). A Tukey multiple comparison corrected post-hoc analysis showed statistically significant difference between R_{Y} and L_{Y} weight magnitudes at Weeks 1 and 3 with a trend towards significance at Week 2 (Week 1, p-value=0.006; Week 2, p-value=0.081; Week 3, p-value=0.004).

### Rotator Cuff Injury Results in Lasting Compensation by the Contralateral Arm.

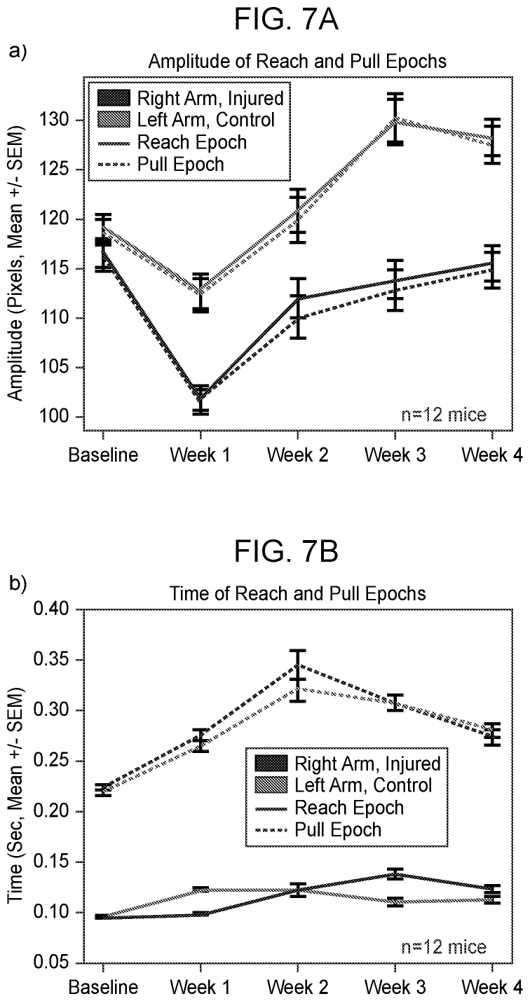

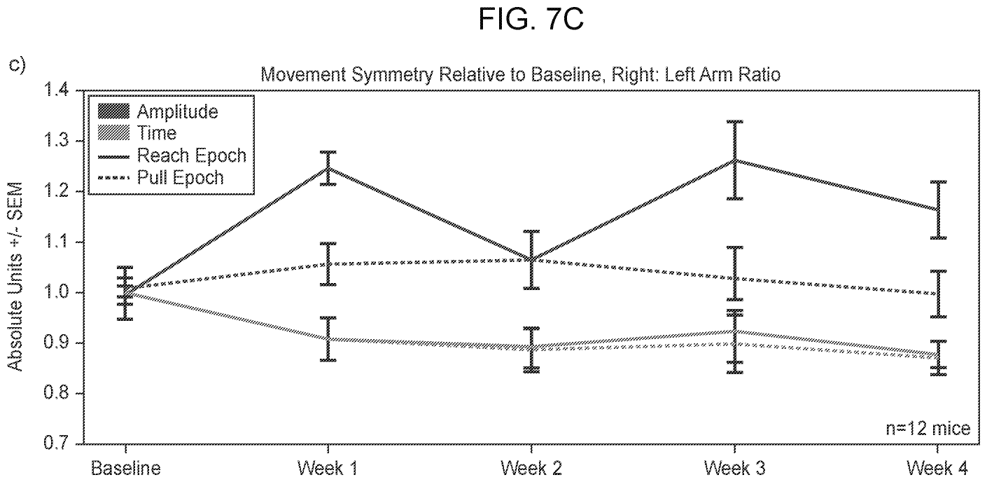

The waveform shape analysis revealed lasting deficits in the reach-to-pull transition while the kinematic synergy analysis shows that while injury caused the temporary emergence of a higher dimensional movement state space, this dimensionality reverted to baseline at the end of four week's recovery. Analyzing movement amplitude (FIG. 7a) showed a striking decrease in movement amplitude of the right arm after rotator cuff injury between Baseline and Week 1. Over the ensuing three weeks of recovery (Week 2 through Week 4), movement amplitude for the injured right arm recovered back to its Baseline while movement amplitude for the left arm exceeded that of the pre-injury baseline, suggesting that mice continue compensating with their left arms even after right arm kinematics recovers. This effect was seen symmetrically across both reaching (FIG. 7a solid lines) and pulling (FIG. 7a dotted lines) epochs. A three-way ANOVA with main effects of time, arm (injured versus uninjured), and movement epoch (reach versus pull) plus an interaction term of time and arm was fit to the amplitude data. Statistically significant main effects of time were found (F(4,5516)=31.601, p-value=4.561*10⁻²⁶) and arm (F(1,5516)=3.456, p-value=6.301*10⁻²) plus a significant effect of the interaction between time and arm (F(4,5516)=10.470, p-value=1.898*10⁻⁸). As expected, there was no significant effect of movement epoch (F(1,5516)=0.583, p-value=0.445). A Tukey multiple comparison corrected post-hoc analysis showed statistically significant differences in the interaction term when comparing Baseline relative to post-injury Weeks for the right arm (Baseline vs Week 1, p-value=1*10⁻³²; Baseline vs Week 2, p-value=0.0639; Baseline vs Week 3, p-value=0.763; Baseline vs Week 4, p-value=0.9993) and for the left arm (Baseline vs Week 1, p-value=0.0008; Baseline vs Week 2, p-value=0.9984; Baseline vs Week 3, p-value=1*10⁻³²; Baseline vs Week 4, p-value=1*10⁻³²). These statistical findings further reinforce how, after just one week's recovery time, the right arm movement amplitude returns back to its pre-injury baseline while the left arm exhibits persistent compensation.

In parallel with quantifying movement amplitude, movement time for reach and pull epochs was quantified (FIG. 7b). Here, a clear difference was noticed between movement epoch with pulls universally taking longer than reaches (FIG. 7b, pull times shown as dotted lines and reach times shown as solid lines). At Baseline both reach and pull times are highly symmetrical. This symmetry is followed by an overall trend to longer reach times for the right arm after rotator cuff injury. Pull times also lengthen, albeit more symmetrically, for both the right and left arms before reaching a peak at Week 2 and then declining for the remaining two weeks. It was hypothesize that the parallel increase in pull times is attributable to different mechanisms for the injured and uninjured arms—the former because injury slows movement and the latter because of compensation which requires the execution of higher amplitude movements. A three-way ANOVA was fitted with main effects of time, arm (injured versus uninjured), and movement epoch (reach versus pull) plus an interaction term of time and arm onto the time data. Statistically significant main effects of time were found (F(4,5516)=55.444, p-value=5.444*10⁻⁴⁶) and movement epoch (F(1,5516)=5101.962, p-value=1*10⁻³²) plus a trend towards a statistically significant effect of the interaction between time and arm (F(4,5516)=2.288, p-value=0.058). There was no significant effect of injured vs uninjured arm (F(1,5516)=0.170, p-value=0.680).

Human Patients with Rotator Cuff Injuries Recapitulate the Kinematic Phenotype Seen in Rodents.

After the precise temporal control over injury afforded by a rodent model was used to develop a clear set of measures that differentiate the kinematics of injured vs uninjured shoulders, the biomarkers were validated on human patients with known RC tears (n=6) as well as healthy controls with no shoulder pathology (n=6). In contrast to mice, whose fur obstructs clear views of the elbow, humans have readily visible elbows in a frontal coronal view and thus the position of both the hands and elbows for human subjects was tracked (FIG. 8a, three cycles of string pulling in a representative control subject). Unrolling the kinematic trace in the Y (vertical)-axis across time revealed striking qualitative similarities in waveform shape across rodents (FIG. 4b) and humans (FIG. 8b, same representative control subject as in 6a with the entire video unrolled across time in the Y-axis): reaches exhibit faster rises as compared to pulls, and both arms oscillate out of phase with respect to each other.

Next, whether waveform shape quantitatively differs across injured vs uninjured shoulders was validated. As in mice, only data for hand movements was used and it was found that patients with injured shoulders exhibited increased full width at half maximum values of the Y-axis string pulling waveform peaks (FIG. 8c, data from control shoulders irrespective of laterality in grey. Data from injured shoulders shown in light tan. Data from contralateral uninjured shoulders shown in orange. Inset provides zoomed view on histogram values on the interval from [0, 100]. Two-sided Kolmogorov-Smirnoff test, control vs injured shoulders: test statistic=0.6652, p-value=1.5*10⁻¹⁷, n=6 controls (12 shoulders), n=6 patients (6 injured shoulders)). In parallel with analyzing waveform shape, the first and second derivatives of the waveform trajectories was taken to analyze velocity and acceleration, respectively. Just as in rodents, a histogram of velocity (in pixels/second) revealed an increased concentration of values around zero (control: 7.853±28.661) for both the injured shoulder (injured: 2.207±13.970) and, curiously, the contralateral uninjured shoulder (contralateral uninjured: 3.782±17.398) in patients with RC tears. Indeed, a one-way Kruskal-Wallis test found a statistically significant main effect of arm (control, injured, and contralateral uninjured) on velocity (test statistic=109.9, p-value=1.37*10⁻²⁴). A Bonferroni-corrected Mann-Whitney U test as a post-hoc analysis showed statistically significant differences in velocity between Control vs Injured (p-value=5.62*10⁻¹⁹), Control vs. Contralateral Uninjured (p-value=6.61*10⁻¹⁷), and Injured vs. Contralateral Uninjured (p-value=0.021). When analyzing acceleration values (in pixels/second²), as a striking of a difference between patients and controls was not observed (control: −153.278±6.921, injured: −77.339±2.916, contralateral uninjured: −97.407±4.463). However, a Kruskal-Wallis test still reached statistical significance (test statistic=21.724, p-value=1.92*10⁻⁵). A Bonferroni-corrected Mann-Whitney U test as a post-hoc analysis showed statistically significant differences in velocity between Control vs Injured (p-value=0.001) and Control vs. Contralateral Uninjured (p-value=5.10*10⁻⁵) but not for Injured vs. Contralateral Uninjured (p-value=1.00).

In contrast to findings in rodents, PCA decomposition of lowpass filtered, mean-centered X/Y position data of the hands and elbows did not reveal a statistically significant difference in dimensionality between control vs RC tear groups (Supp. XYZ; main effect of control vs RC tear group, F(1,48)=1.437, p-value=0.237; main effect of PC number, F(1,48)=707.744, p-value=2.195*10⁻³⁰; interaction between group and PC number, F(1,48)-1.927, p-value=0.171; statistics performed only on the first two PCs as they cumulatively explain >90% of the variance in the data). However, there was a trend towards statistical significance in the analysis of Eigenvector magnitudes (FIG. 8e) for Y-axis movements of the hands across the control shoulder (−0.010±0.138), injured shoulders (−0.273±0.133), and contralateral uninjured shoulders (0.218±0.169), one-way ANOVA main effect of group F(2,49)=2.578, p-value=0.086.

When longitudinally testing injured mice on the string pulling task, persistent compensation by the contralateral, uninjured extremity was noticed that persists even as the kinematics of the injured extremity recovers (FIG. 7ai, 7aii). The same metrics of amplitude and time were analyzed for the human subjects. It was found that there was no statistically significant difference in movement amplitude (FIG. 8f) for reach and pull epochs across all control, injured, and contralateral uninjured shoulders nor was there a statistically significant interaction between reach vs pull and group (two-way ANOVA, main effect of epoch, F(1,502)=0.179, p-value=0.672; interaction of epoch and group, F(2,502)=0.177, p-value=0.838). Mean amplitude in pixels±SEM for control group (reach: 552.458±16.834, pull: 541.680±16.708), injured (reach: 441.589±19.191, pull: 450.136±21.940), and contralateral uninjured (reach: 510.682±20.509, pull: 498.566±21.012). There was, however, a statistically significant main effect of group on amplitude (two-way ANOVA, main effect of group, F(2,502)=5.674, p-value=0.004). A Tukey multiple comparison corrected post-hoc analysis showed statistically significant differences in the main effect of group for Control vs Injured (p-value=2.744*10⁻⁷) and Injured vs Contralateral Uninjured (p-value=8.359*10⁻³) but not for Control vs Contralateral Uninjured (p-value=0.067). Together, these results strongly suggest that patients with RC injury have reduced movement amplitude of the injured extremity.

When analyzing the timing of reach and pull epochs (FIG. 8g, outlier points >4 seconds removed for clarity. See Supp. XYZ for plot with all data points shown), it was noticed that pulls (dashed line) generally take longer than reaches—a replication of the phenomenon that was seen in the rodent data (FIG. 7aii, dashed lines). Mean time in seconds±SEM for control group (reach: 0.601±0.026, pull: 0.993±0.055), injured (reach: 1.383±0.118, pull: 1.788±0.152), and contralateral uninjured (reach: 1.087±0.081, pull: 2.169±0.216). A two-way ANOVA revealed a statistically significant main effect of epoch (reach vs pull, F(1,502)=6.543, p-value=0.011) as well group (control, injured, and contralateral uninjured, F(2,502)=27.122, p-value=6.533*10⁻¹²); the interaction between epoch and group was also statistically significant (F(2,502)=5.524, p-value=0.004). A Tukey multiple comparison corrected post-hoc analysis showed statistically significant differences in the interaction between group and epoch for control group pull time vs injured group pull times (p-value=2.357*10⁻⁵), control group pull times and contralateral uninjured group pull times (p-value=5.303*10⁻¹¹), injured group pull times and control group reach times (p-value=1.822*10⁻¹¹), injured group pull times and contralateral uninjured group reach times (p-value=6.635*10⁻⁴), contralateral uninjured group pull times and control group reach times (p-value=1.029*10⁻¹³), contralateral uninjured group pull times and injured group reach times (p-value=5.355*10⁻⁵), and contralateral uninjured group pull times and contralateral uninjured group reach times (p-value=1.006*10⁻⁸). Curiously, it was noticed that the contralateral uninjured arm pull time is greater than the contralateral uninjured arm reach time. The divergence in these two measures is striking given the downward trend in reach times for the contralateral uninjured arm vs both reach and pull times for the injured arm. While difficult to ascertain with certainty, it is suspected that this may be a manifestation of compensation where, in order to advance the string by an equivalent amplitude during reach and pull epochs (FIG. 8f), patients are recruiting scapular or thoracic motions thus prolonging the movement cycle.

As a final analysis, the dynamic range ratio was compared, computed by taking the ratio of standard deviation values of the lowpass filtered, mean-centered Y-axis kinematic trajectory between ipsilateral hand:elbow pairs across all study participants (FIG. 8h). Determining whether rotator cuff injury predisposes patients to adopting a movement regime where the string is advanced by rotating the humerus around its longitudinal axis vs engaging the entire arm in reaching & pulling motion was of interest. In other words, it was expected that dynamic range ratio would increase for patients that predominately advance the string by moving their hands using rotational (rather than translational reaching) movements while keeping the elbow stationary. Indeed, a statistically significant increase in the movement dynamic range ratio was noticed (one-way ANOVA, main effect of group, F(2,49)=5.404, p-value=0.008; Tukey multiple comparison corrected post-hoc revealed a significant difference between control and injured extremities, p-value=0.006). The mean standard deviation±SEM across groups was 1.665±0.069 for control extremities, 3.224±0.525 for injured extremities, and 2.613±0.373 for contralateral uninjured extremities.

### Discussion

In summary, it was found that the string pulling task was a sensitive assay for assessing both human and rodent shoulder injury. After injury to the SS/IS tendons, mice exhibited changes in quantitative measures of waveform shape (FIGS. 4 and 5) that persisted for the duration of the experiment even as straightforward kinematic measures (such as movement amplitude) were restored back their pre-injury baseline (FIG. 7). Disruptions in cross-frequency coupling of kinematic synergies across injury and recovery as well as the emergence of a higher dimensional kinematic state space after injury was also noticed (FIG. 6). Critically, nearly all of the pre-clinical biomarkers translated directly to human patients and exhibited similar phenotypic shifts with RC injury (FIG. 8).

### String Pulling as a Novel Animal Model of Shoulder Function.

Previous animal models of rodent tendon injury and repair have invariably drawn inferences about upper or lower extremity function using quadruped gait tasks. In these models, animal subjects are tasked with walking either on a transparent treadmill or bromophenol blue coated paper and the resultant paw prints are scored for measures such as stride length, stride width, paw print area, paw length, or toe spread [24-28]. More recent techniques have included force sensor measurement as rats walk through a transparent treadmill [29]. While these methods have demonstrated functional differences between subtypes of RC tendon injury [25] or RC tendon repair strategies [28], [29], no study has ever analyzed forelimb function in rodents using bimanual forelimb movements that are analogous to the types of motions that humans perform in daily life. The string pulling assay has multiple advantages over quadruped gait tasks: 1) it allows for the kinematic assessment of each arm independently (and outside the gait cycle) thus allowing for a within-animal control using the contralateral extremity, 2) the task includes a significant overhead motion component which is frequently impaired in patients with RC tears and that challenges the supraspinatus muscle (one of the most commonly injured muscles in the RC), 3) movements of the lower extremities are decoupled from movements of the arms, and 4) existing literature, plus generated experimental results, suggest strong kinematic concordance between rodents and humans in string pulling task performance [12]. Moreover, the assay is inexpensive requiring only a piece of string, a transparent box (for rodent experiments), and a smartphone capable of video recording. Image segmentation of the resulting videos using deep convolutional neural networks may now be easily accomplished on computers equipped with consumer-grade Graphical Processing Units (GPUs) [19].

Waveform Shape and Kinematic Synergy Analyses Provide Latent Insights into Movement Quality.

Waveform shape: even though a clear difference was observed in string pulling movement speed and amplitude between injured and uninjured extremities in both rodents and humans (FIGS. 7 and 8), the oscillatory nature of the behavior allows for application of inter-disciplinary time series analysis techniques. For example, measures of waveform shape have recently been identified in the motor neuroscience literature as potential biomarkers present in neural signals recorded from the brains of patients with Parkinson's disease [30]. Here, the full width at half maximum (FWHM) for waveform peaks (i.e., capturing the reach-to-pull transition) of Y-axis string pulling kinematic trajectories was analyzed. Analysis was focused on the reach-to-pull transition as it 1) captures the overhead reaching movement as it requires the supraspinatus muscle (which were iatrogenically injured in the rodent model) and 2) it highlights the transition from a movement regime requiring no force application (reaching) to a movement regime requiring force application (pulling), especially in the mouse model where the forces being applied are relatively high given the animals' body weight. Importantly, FWHM is not simply co-linear with movement duration: in FIG. 7aii it was noticed that both injured and uninjured arms have prolonged pull durations after injury yet the distribution of FWHM undergoes a rightward shift (i.e., left skewed) only for those animals with RC injury (FIG. 5a). While further research into this phenomenon is required, it is suspected that FWHM is a surrogate measure for neuromuscular control of the reach-to-pull transition. In animals and humans with RC injury, altered proprioceptive input from the injured tendon, or nociceptive input from the joint itself, necessitates increased top-down control over the shoulder's musculature from the central nervous system to switch movement regimes.

Kinematic synergies: Prior human research suggests that the neural control of high degree of freedom (hDOF) joints (such as the hand) is encoded via a low dimensional neural and kinematic manifold underlying motor movements [20], [21], [31], [32]. This manifold captures a low-dimensional kinematic basis set—termed as a synergy—to simplify control of hDOF joints. Analogous to the hand, the shoulder joint occupies a high dimensional anatomic space with 8 degrees of motion and 18 different muscles controlling its articulation. Intriguingly, for human string pulling behavior (where humans have access to an eight-dimensional kinematic space from the X/Y position data for four joints), only two principal components are sufficient to explain >90% of the variance in the data (see Supp. XYZ, red line shows mean cumulative variance±SEM for all patients). This suggests that the brain encodes a low dimensional kinematic manifold that smoothly covaries shoulder flexors, extensors, and stabilizers to generate a smooth, sinusoidal movement. Moreover, no prior research has been done studying the effects of musculoskeletal injury and recovery on kinematic synergies of the shoulder. In rodents, where there is ready access to longitudinal data, an increase in the dimensionality of the kinematic state-space after injury which recovers back to baseline at the end of four weeks' recovery was noticed. The cross-frequency coupling between PC1 and PC2 undergoes a similar pattern. While access to neural or electromyographic (EMG) data was not achieved, this work provides a robust injury model for probing these substrates in future studies.

### Empowering Computerized Physical Therapy at Scale.

Here inexpensive assay that is highly sensitive for assessing shoulder function across species was developed. Both the mouse and human video recordings were collected using a smartphone. Given the rise of matrix math “coprocessor” chips embedded into recent-generation smartphone devices, it is now possible to perform image segmentation and analysis entirely on a smartphone device without uploading video over the internet. By deploying the simple metrics calculated by the string pulling task through a user-facing smartphone app, privacy-preserving measurements of shoulder health to the community can be provided. Patients could then use such measures to track their recovery from shoulder injury or surgery. Indeed, systems where the metrics calculated on sinusoidal signals derived from simple physical tasks are used to diagnose MSK injuries in other areas, such as the back, hip, or knee can be developed.

### REFERENCES

1. Symptoms Matter-Leading Causes of Disability. NCCIH https://www.nccih.nih.gov/about/symptoms-matterleading-causes-of-disability.
2. Brukner, P. & Khan, K. Brukner & Khan's clinical sports medicine. Volume 1: Injuries. (McGraw-Hill Education (Australia), 2017).
3. Freburger, J. K. & Holmes, G. M. Physical Therapy Use by Community-Based Older People. Phys. Ther. 85, 19-33 (2005).
4. Arnadottir, S. A. & Jonsson, B. G. Outpatient physical therapy population has been aging faster than the general population: a total population register-based study. BMC Health Serv. Res. 21, 708 (2021).
5. Urwin, M. et al. Estimating the burden of musculoskeletal disorders in the community: the comparative prevalence of symptoms at diVerent anatomical sites, and the relation to social deprivation. 7 (1998).
6. Terry, G. C. & Chopp, T. M. Functional Anatomy of the Shoulder. J. Athl. Train. 35, 248-255 (2000).
7. Minagawa, H. et al. Prevalence of symptomatic and asymptomatic rotator cuff tears in the general population: From mass-screening in one village. J. Orthop. 10, 8-12 (2013).
8. Tempelhof, S., Rupp, S. & Seil, R. Age-related prevalence of rotator cuff tears in asymptomatic shoulders. J. Shoulder Elbow Surg. 8, 296-299 (1999).
9. Liu, X. et al. A Mouse Model of Massive Rotator Cuff Tears. JBJS 94, e41 (2012).
10. Liu, X. et al. Investigating the cellular origin of rotator cuff muscle fatty infiltration and fibrosis after injury. Muscles Ligaments Tendons J. 6, 6-15 (2016).
11. Blackwell, A. A., Banovetz, M. T., Qandeel, Whishaw, I. Q. & Wallace, D. G. The structure of arm and hand movements in a spontaneous and food rewarded on-line string-pulling task by the mouse. Behav. Brain Res. 345, 49-58 (2018).
12. Singh, S. et al. Human string-pulling with and without a string: movement, sensory control, and memory. Exp. Brain Res. 237, 3431-3447 (2019).
13. Schwartz, C., Hazée, A., Denoël, V. & Brüls, O. SHOULDER INJURY PREVENTION IN SPORTS USING 3D MOTION CAPTURE. 1.
14. Rawashdeh, S. A., Rafeldt, D. A., Uhl, T. L. & Lumpp, J. E. Wearable motion capture unit for shoulder injury prevention. in 2015 IEEE 12th International Conference on Wearable and Implantable Body Sensor Networks (BSN) 1-6 (IEEE, 2015). doi:10.1109/BSN.2015.7299417.
15. Gritsenko, V. et al. Feasibility of Using Low-Cost Motion Capture for Automated Screening of Shoulder Motion Limitation after Breast Cancer Surgery. PLOS ONE 10, e0128809 (2015).
16. Park, C. et al. Comparative accuracy of a shoulder range motion measurement sensor and Vicon 3D motion capture for shoulder abduction in frozen shoulder. Technol. Health Care 30, 251-257 (2022).
17. Jackson, M., Michaud, B., Tétreault, P. & Begon, M. Improvements in measuring shoulder joint kinematics. J. Biomech. 45, 2180-2183 (2012).
18. Charbonnier, C., Chagué, S., Kolo, F. C., Chow, J. C. K. & Lädermann, A. A patient-specific measurement technique to model shoulder joint kinematics. Orthop. Traumatol. Surg. Res. 100, 715-719 (2014).
19. Mathis, A. et al. DeepLabCut: markerless pose estimation of user-defined body parts with deep learning. Nat. Neurosci. 21, 1281-1289 (2018).
20. Natraj, N., Silversmith, D. B., Chang, E. F. & Ganguly, K. Compartmentalized dynamics within a common multi-area mesoscale manifold represent a repertoire of human hand movements. Neuron 110, 154-174.e12 (2022).
21. Ejaz, N., Hamada, M. & Diedrichsen, J. Hand use predicts the structure of representations in sensorimotor cortex. Nat. Neurosci. 18, 1034-1040 (2015).
22. Safavynia, S., Torres-Oviedo, G. & Ting, L. Muscle Synergies: Implications for Clinical Evaluation and Rehabilitation of Movement. Top. Spinal Cord Inj. Rehabil. 17, 16-24 (2011).
23. Understanding phase-amplitude coupling from bispectral analysis. 17.
24. Soslowsky, L. J., Carpenter, J. E., DeBano, C. M., Banerji, I. & Moalli, M. R. Development and use of an animal model for investigations on rotator cuff disease. J. Shoulder Elbow Surg. 5, 383-392 (1996).
25. Perry, S. M., Getz, C. L. & Soslowsky, L. J. Alterations in function after rotator cuff tears in an animal model. J. Shoulder Elbow Surg. 18, 296-304 (2009).
26. Messner, K., Wei, Y., Andersson, B., Gillquist, J. & Räsänen, T. Rat Model of Achilles Tendon Disorder. Cells Tissues Organs 165, 30-39 (1999).
27. Fu, S.-C., Chan, K.-M., Chan, L.-S., Fong, D. T.-P. & Lui, P.-Y. P. The use of motion analysis to measure pain-related behaviour in a rat model of degenerative tendon injuries. J. Neurosci. Methods 179, 309-318 (2009).
28. Wang, Z. et al. A Mouse Model of Delayed Rotator Cuff Repair Results in Persistent Muscle Atrophy and Fatty Infiltration. Am. J. Sports Med. 46, 2981-2989 (2018).
29. Sarver, J. J., Dishowitz, M. I., Kim, S.-Y. & Soslowsky, L. J. Transient Decreases in Forelimb Gait and Ground Reaction Forces Following Rotator Cuff Injury and Repair in a Rat Model. J. Biomech. 43, 778-782 (2010).
30. Cole, S. R. et al. Nonsinusoidal Beta Oscillations Reflect Cortical Pathophysiology in Parkinson's Disease. J. Neurosci. 37, 4830-4840 (2017).
31. Santello, M., Flanders, M. & Soechting, J. F. Postural Hand Synergies for Tool Use. J. Neurosci. 18, 10105-10115 (1998).
32. Bizzi, E., Cheung, V. C. K., d'Avella, A., Saltiel, P. & Tresch, M. Combining modules for movement. Brain Res. Rev. 57, 125-133 (2008).
33. Aarts, E., Verhage, M., Veenvliet, J. V., Dolan, C. V. & van der Sluis, S. A solution to dependency: using multilevel analysis to accommodate nested data. Nat. Neurosci. 17, 491-496 (2014).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims. The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of generating a mobility assessment for a subject, the method comprising:
instructing the subject to perform an activity comprising an oscillatory motion;
generating a visual recording of the subject performing the activity using a recording device;
extracting time series data from the visual recording using a dynamic algorithm;
generating one or more musculoskeletal movement biomarkers from the time series data;
producing the mobility assessment for the subject from the one or more musculoskeletal movement biomarkers.

2. The method according to claim 1, wherein the oscillatory motion is repeated 5 or more times.

3. The method according to claim 2, wherein the oscillatory motion is repeated 10 or more times.

4. The method according to any of the preceding claims, wherein the oscillatory motion comprises the movement of a joint of the subject.

5. The method according to claim 4, wherein the joint is a ball and socket joint.

6. The method according to any of the preceding claims, wherein the oscillatory motion comprises the repeated abduction, adduction, flexion, extension, or circumduction of one or more body parts of the subject.

7. The method according to claim 6, wherein the oscillatory motion comprises the repeated abduction or adduction of one or more body parts of the subject.

8. The method according to claim 6, wherein the oscillatory motion comprises the repeated flexion and extension of one or more body parts of the subject.

9. The method according to any of claims 6 to 8, wherein the one or more body parts comprises the subjects arms, legs, pelvis, hips, back, thorax, or shoulders.

10. The method according to claim 9, wherein the oscillatory motion comprises movement of the subjects shoulders.

11. The method according to claim 10, wherein the oscillatory motion comprises movement of the subjects shoulder girdles.

12. The method according to claim 11, wherein the oscillatory motion comprises the vertical pulling of a string or rope.

13. The method according to claim 9, wherein the oscillatory motion comprises movement of the subjects legs or hips.

14. The method according to claim 13, wherein the oscillatory motion comprises a crab walk or a monster walk.

15. The method according to claim 13, wherein the oscillatory motion comprises movement of the subjects knees.

16. The method according to claim 13 or 15, wherein the oscillatory motion comprises lateral lunges, forward lunges, reverse lunges, or deadlifts.

17. The method according to claim 16, wherein the oscillatory motion comprises alternating single leg reverse deadlifts.

18. The method according to claim 13, wherein the oscillatory motion comprises movement of the subjects ankles.

19. The method according to claim 18, wherein the oscillatory motion comprises heel raises.

20. The method according to claim 9, wherein the oscillatory motion comprises movement of the subjects back.

21. The method according to claim 20, wherein the oscillatory motion comprises back extensions, side bending, forward bending, or squats.

22. The method according to any of the preceding claims, wherein the visual recording is generated without the use of a motion tracking marker.

23. The method according to any of claims 1 to 21, wherein the visual recording comprises the use of a motion tracking marker or sensor.

24. The method according to claim 23, wherein the motion tracking marker or sensor is a smartwatch.

25. The method according to claim 23 or 24, wherein the motion tracking marker or sensor comprises a visual pattern or emits an audio frequency.

26. The method according to claim 25, wherein the visual pattern or audio frequency is used to determine a distance between the recording device and the motion tracking marker or sensor.

27. The method according to claim 25, wherein the visual pattern or audio frequency is used to determine a speed at which the motion tracking marker or sensor is moving toward or away from the recording device.

28. The method according to any of the preceding claims, wherein the method further comprises positioning the subject a distance from the recording device.

29. The method according to claim 28, wherein the positioning is based on feedback generated by the automatic detection of one or more body landmarks of the subject.

30. The method according to claim 29, wherein the body landmark comprises facial features of the subject.

31. The method according to claim 28, wherein the positioning is based on feedback generated by the automatic detection of a motion tracking marker or sensor.

32. The method according to any of the preceding claims, wherein the recording device is configured to emit a laser beam.

33. The method according to claim 32, wherein the laser beam is a vertical-cavity surface-emitting laser beam.

34. The method according to claim 32 or 33, wherein the recording device comprises a LiDAR scanner.

35. The method according to claim 34, wherein the method further comprises positioning the subject a distance from the recording device based on feedback from the LiDAR scanner.

36. The method according to any of the preceding claims, wherein the recording device is configured to generate a sequence of visual images over time.

37. The method according to claim 36, wherein the recording device is a webcam or smartphone.

38. The method according to claim 37, wherein the visual recording is generated at 15 or more frames per second.

39. The method according to any of the preceding claims, wherein the time series data comprises the location of one or more body parts of the subject.

40. The method according to claim 39, wherein the time series data comprises a waveform generated by graphing the location of a body part of the subject on an axis over time.

41. The method according to claim 40, wherein the axis is the vertical axis.

42. The method according to claim 40, wherein the axis is the horizontal axis.

43. The method according to claim 40, wherein the axis is the depth axis.

44. The method according to any of the preceding claims, wherein the dynamic algorithm comprises a machine learning algorithm.

45. The method according to claim 44, wherein the machine learning algorithm comprises a neural network.

46. The method according to claim 45, wherein the neural network is a convolutional neural network or a recurrent neural network.

47. The method according to claim 45 or 46, wherein the neural network comprises a ResNet, InceptionNet, VGGNet, GoogLeNet, AlexNet, EfficientNet, or YOLONet neural network.

48. The method according to any of claims 45 to 47, wherein the neural network is 10 or more layers deep.

49. The method according to any of claims 44 to 48, wherein the dynamic algorithm is trained using DeepLabCut™, DeepPoseKit, LEAP, SLEAP, or Anipose.

50. The method according to any of claims 44 to 49, wherein the dynamic algorithm is trained using an ImageNet, COCO, OID, or PASCAL data set.

51. The method according to any of the preceding claims, wherein the extracted time series data is filtered.

52. The method according to claim 51, wherein the extracted time series data is filtered using a high pass filter.

53. The method according to claim 51 or 52, wherein the extracted time series data is filtered using a low pass filter.

54. The method according to any of claims 51 to 53, wherein the extracted time series data is filtered using a signal processing filter.

55. The method according to claim 54, wherein the extracted time series data is filtered using a Butterworth, Chebyshev, Elliptic, or Linkwitz-Riley filter, Savitzky-Golay filter.

56. The method according to any of the preceding claims, wherein one or more of the musculoskeletal movement biomarkers is selected from the group consisting of oscillatory motion amplitude, duration, full width at half maximum, acceleration, and velocity.

57. The method according to any of the preceding claims, wherein one or more of the musculoskeletal movement biomarkers is generated using principal component analysis.

58. The method according to claim 57, wherein one or more of the musculoskeletal movement biomarkers is generated by comparing different principal components of a principal component analysis.

59. The method according to claim 58, wherein the comparison is performed using bispectral coherence analysis.

60. The method according to any of the preceding claims, wherein one or more of the musculoskeletal movement biomarkers is related to the symmetry of the oscillatory motion.

61. The method according to claim 60, wherein the symmetry is between abduction and adduction movements.

62. The method according to claim 60, wherein the symmetry is between reaching and pulling movements.

63. The method according to claim 60, wherein the symmetry is between flexion and extension movements.

64. The method according to claim 60, wherein the symmetry is between arclengths of one or more circumduction movements.

65. The method according to any of the preceding claims, wherein one or more of the musculoskeletal movement biomarkers is related to the correlation between separate body parts performing the oscillatory motion.

66. The method according to claim 65, wherein the separate body parts perform the oscillatory motion concurrently.

67. The method according to claim 65, wherein the separate body parts perform the oscillatory motion at separate times.

68. The method according to claim 66 or 67, wherein the separate body parts are equivalent bilaterally symmetric body parts.

69. The method according to claim 66 or 67, wherein the separate body parts are bilaterally asymmetric to each other.

70. The method according to any of the preceding claims, wherein one or more of the musculoskeletal movement biomarkers is a movement dynamic range ratio between two body parts.

71. The method according to any of the preceding claims, wherein the mobility assessment comprises a quantitative score of movement quality.

72. The method according to claim 71, wherein the quantitative score is a composite of two or more musculoskeletal movement biomarkers.

73. The method according to any of the preceding claims, wherein the mobility assessment comprises the diagnosis of a disease or condition.

74. The method according to any of the preceding claims, wherein the mobility assessment comprises a determination regarding whether one or more body parts is being compensated for.

75. The method according to any of the preceding claims, wherein the mobility assessment comprises a determination regarding whether one or more body parts is compensating for another body part.

76. The method according to any of the preceding claims, wherein the mobility assessment comprises an assessment of the subject's fitness for performing a task.

77. The method according to claim 76, wherein the task is a dynamic open or closed kinetic chain activity.

78. The method according to claim 77, wherein the task is a weightlifting or strength training movement.

79. The method according to any of the preceding claims, wherein the visual recording is generated at two or more timepoints to generate two or more mobility assessments.

80. The method according to claim 79, wherein the two or more timepoints are at least a minute apart from each other.

81. The method according to claim 80, wherein the two or more timepoints are at least a month apart from each other.

82. The method according to claim 79 or 81, wherein a first timepoint of the two or more timepoints occurs after an injury of the subject.

83. The method according to claim 79 or 81, wherein a first timepoint of the two or more timepoints occurs before an injury of the subject.

84. The method according to claim 83, wherein a subsequent timepoint occurs after an injury of the subject.

85. The method according to claim 79 or 81, wherein a first timepoint of the two or more timepoints occurs after the subject has received a medical intervention.

86. The method according to claim 79 or 81, wherein a first timepoint of the two or more timepoints occurs before the subject has received a medical intervention.

87. The method according to claim 86, wherein a subsequent timepoint occurs after the subject has received a medical intervention.

88. The method according to claim 79 or 81, wherein the subject has not received medical intervention.

89. The method according to any of claims 79 to 88, wherein the two or more generated mobility assessments are used to determine a level of recovery of the subject after an injury.

90. The method according to any of claims 79 to 87, wherein the two or more generated mobility assessments are used to determine a level of recovery of the subject after a surgery.

91. The method according to any of claims 79 to 87, wherein the two or more generated mobility assessments are used to determine a level of effectiveness of a medical intervention.

92. The method according to any of claims 79 to 91, wherein the two or more generated mobility assessments are used to determine a decline in the mobility of the subject.

93. The method according to any of the preceding claims, wherein the subject is a human.

94. The method according to claim 93, wherein the human has a disease or condition.

95. The method according to claim 94, wherein the disease or condition is arthritis.

96. The method according to claim 94, wherein the disease or condition is tendonitis.

97. The method according to claim 94, wherein the disease or condition is a tendon or myotendinous tear.

98. The method according to claim 94, wherein the disease or condition is a hernia.

99. The method according to claim 93, wherein the human is 60 years of age or older.

100. The method according to claim 93, wherein the human is younger than 60 years of age.

101. The method according to claim 93, wherein the human has experienced an injury.

102. The method according to claim 101, wherein the injury is a musculoskeletal injury.

103. The method according to claim 102, wherein the injury is an injury of the shoulder.

104. The method according to claim 103, wherein the injury is an injury of the rotator cuff.

105. The method according to any of claims 101 to 104, wherein the injury is a muscle strain or a muscle tear.

106. The method according to claim 101, wherein the injury is a sprain.

107. The method according to any of claims 101 to 106, wherein the injury has occurred in the last year.

108. The method according to any of claims 101 to 106, wherein the injury has occurred a year or more in the past.

109. The method according to claim 93, wherein the human regularly performs physical training exercises.

110. The method according to claim 93, wherein the human has received surgery.

111. The method according to claim 110, wherein the surgery occurred on the back, a knee, a hip, an ankle, or a shoulder.

112. The method according to claim 110 or 111, wherein the surgery occurred in the last year.

113. The method according to claim 110 or 111, wherein the surgery occurred a year or more in the past.

114. The method according to any of the preceding claims, wherein the mobility assessment is produced at least in part using a dynamic algorithm.

115. The method according to any of the preceding claims, wherein the mobility assessment is saved to a database.

116. The method according to claim 115, wherein the database is used to determine a relationship between health outcomes and one or more musculoskeletal movement biomarkers.

117. The method according to claim 115, wherein the database is used to determine a relationship between the diagnosis of a disease or condition and one or more musculoskeletal movement biomarkers.

118. The method according to claim 115, wherein the database is used to determine a relationship between the fitness of a subject for performing a task and one or more musculoskeletal movement biomarkers.

119. The method according to any of claim 116 or 118, wherein the relationship is determined at least in part using a dynamic algorithm.

120. The method according to claim 119, wherein the dynamic algorithm is a machine learning algorithm.

121. The method according to any of claims 116 to 120, wherein the determined relationship is used to generate subsequent mobility assessments.

122. The method according to any of the preceding claims, wherein the mobility assessment is produced using a computer or smartphone.

123. The method according to claim 122, wherein the mobility assessment is produced using a computer or smartphone app.

124. A mobility analysis system configured to perform the method according to any of claims 1 to 123.

125. A system for generating a mobility assessment for a subject, the system comprising:
a display configured to provide visual information instructing the subject to perform an activity comprising an oscillatory motion;
a digital recording device configured to generate a visual recording of the subject performing the activity comprising an oscillatory motion;
a processor configured to receive the visual recording generated by the camera; and
memory operably coupled to the processor wherein the memory comprises instructions stored thereon, which when executed by the processor, cause the processor to extract time series data from the visual recording using a dynamic algorithm, generate one or more musculoskeletal movement biomarkers from the time series data, and produce the mobility assessment for the subject from the one or more musculoskeletal movement biomarkers.

126. The system according to claim 125, wherein the visual information comprises instructions for performing an activity comprising an oscillatory motion.

127. The system according to claim 125 or 126, wherein the system further comprises a speaker configured to provide audio information to the subject.

128. The system according to claim 127, wherein the audio information comprises instructions for performing an activity comprising an oscillatory motion.

129. The system according to any of claims 125 to 128, wherein the digital recording device is configured to generate a sequence of visual images over time.

130. The system according to claim 129, wherein the digital recording device is a webcam or smartphone.

131. The system according to claim 130, wherein the webcam or smartphone is configured to generate a visual recording at a rate of at least 30 frames per second.

132. The system according to any of claims 125 to 131, wherein the system further comprises a device configured to guide the movement of one or more body parts of the subject in performing the oscillatory motion.

133. The system according to claim 132, wherein the device comprises a string or rope for performing the oscillatory motion.

134. The system according to claim 133, wherein the string or rope is configured to be vertically pulled.

135. The system according to any of claims 125 to 134, wherein the system further comprises a motion tracking marker or sensor.

136. The system according to claim 135, wherein the motion tracking marker or sensor is a smartwatch.

137. The system according to claim 135 or 136, wherein the motion tracking marker or sensor comprises a visual pattern or emits an audio frequency.

138. The system according to claim 137, wherein the digital recording device is configured to generate one or more visual images comprising the visual pattern.

139. The system according to claim 138, wherein the memory comprises instructions stored thereon, which when executed by the processor, cause the processor to determine a distance between the recording device and the motion tracking marker or sensor using the one or more visual images comprising the visual pattern.

140. The system according to claim 137, wherein the system further comprises an audio recording device configured to generate one or more data signals based on measurements of the audio frequency.

141. The system according to claim 140, wherein the memory comprises instructions stored thereon, which when executed by the processor, cause the processor to determine a speed at which the motion tracking marker or sensor is moving toward or away from the recording device using the one or more audio frequency data signals.

142. The system according to any of claims 125 to 141, wherein the visual information comprises instructions for positioning the subject a distance from the recording device.
